(19) United States
(12) Patent Application Publication
(10) Pub. No.: US 2014/0283225 A1
(43) Pub. Date: Sep. 18, 2014

(54) EARLY DETECTION AND ELIMINATION OF NON GERM-LINE EVENTS IN THE SOYBEAN TRANSFORMATION PROCESS

(71) Applicant: Dow Agrosciences LLC, Indianapolis, IN (US)

(72) Inventors: Jayakumar Pon Samuel, Carmel, IN (US); Sivarama Reddy Chennareddy, West Lafayette, IN (US); Andy Bowling, Zionsville, IN (US); John Whitteck, Carmel, IN (US); Tatyana Minnicks, Pittsboro, IN (US); Dayakar Pareddy, Carmel, IN (US); Kerrm Y. Yau, Carmel, IN (US); Kelley A. Smith, Lebanon, IN (US)

(73) Assignee: Dow Agrosciences LLC, Indianapolis, IN (US)

(21) Appl. No.: 14/211,091

(22) Filed: Mar. 14, 2014

Related U.S. Application Data
(60) Provisional application No. 61/789,379, filed on Mar. 15, 2013.

(51) Int. Cl. G01N 33/569

(52) U.S. Cl. CPC G01N 33/56961
USPC 800/312; 435/7.92

(57) ABSTRACT

The present disclosure relates in part to a method for identifying a soybean germline transformant from a population of soybean transformants which are comprised of a combination of soybean non-germline transformants and soybean germline transformants. The soybean non-germline transformants are identified and eliminated early in the transformation process. The soybean germline transformants are detected and selected for culturing into mature soybean plants. The method is readily applicable for screening and obtaining a soybean germline transformant at an early stage in the transformation process.

### CROSS-REFERENCE TO RELATED APPLICATION

This application claims the benefit of U.S. Provisional Patent Application Ser. No. 61/789,379, filed Mar. 15, 2013, the disclosure of which is hereby incorporated herein in its entirety by this reference.

### REFERENCE TO SEQUENCE LISTING SUBMITTED ELECTRONICALLY

The official copy of the sequence listing is submitted electronically via EFS-Web as an ASCII formatted sequence listing with a file named “70524SEQUENCES,” created on Mar. 11, 2013, and having a size of 9 kilobytes and is filed concurrently with the specification. The sequence listing contained in this ASCII formatted document is part of the specification and is herein incorporated by reference in its entirety.

### TECHNICAL FIELD

The present disclosure relates in part to a method for identifying a soybean germline transformant from a population of soybean transformants which are comprised of a combination of soybean non-germline transformants and soybean germline transformants. Accordingly, the soybean non-germline transformants are identified and eliminated early in the transformation process. The soybean germline transformants are detected and selected for culturing into mature soybean plants. The method is readily applicable for screening and obtaining a soybean germline transformant at an early stage in the transformation process.

### BACKGROUND

Soybean transformation methodologies have been developed and improved over the last thirty years. This evolution of soybean transformation methodologies has resulted in the capability to successfully introduce a transgene comprising an agronomic trait within the soybean genome. The introduction of herbicide tolerant transgenic soybean events in the mid-1990s, provided producers with a new and convenient technological innovation for controlling a wide spectrum of weeds, which was unparalleled in cultivation farming methods. Currently, transgenic soybeans are commercially available throughout the world, and new transgenic soybean products such as ENLIST™ Soybeans offer improved solutions for ever-increasing weed challenges. The utilization of transgenic soybeans in modern agronomic practices would not be possible but for the development and improvement of soybean transformation methodologies. New and improved soybean transformation methodologies that can be utilized to detect and select soybean germline transformants are essential for meeting the challenges that novel, complex agronomic traits require for introgression within the soybean genome.

The early identification and selection of soybean germline transformants in a transformation process is preferred as the soybean germline transformants comprise a stably integrated transgene which is heritable in subsequent generations. Because the transformation process is relatively inefficient, large numbers of transformants must be produced to identify and select the preferred soybean germline transformants from undesirable soybean non-germline transformants. Many of the transformants obtained from the transformation process are chimeric or soybean non-germline transformants. On average, about 40 to 70 percent of all isolated transformants are soybean non-germline transformants. These soybean non-germline transformants are undesirable and are culled. But this culling occurs only after the transformants have been maintained throughout the transformation process and have advanced to maturity. Maintaining undesirable transformants, e.g., non-germline soybean transformants, results in an inefficient use of resources. Great costs are expended in producing transgenic plants. Such costs exceed pecuniary concerns and include the use of scientists' time, materials, and laboratory space.

The development and improvement of a method that can be utilized to screen and detect soybean germline transformants at the initial stages of the plant transformation is highly desirable as the method can improve the efficiency of the transformation process.

The foregoing examples of the related art and limitations related therewith are intended to be illustrative and not exclusive. Other limitations of the related art will become apparent to those of skill in the art upon a reading of the specification.

### DISCLOSURE

In a preferred embodiment, the disclosure relates to a method for identifying a soybean germline transformant comprising the steps of: transforming a soybean plant tissue with a transgene; regenerating a shoot from the transformed soybean plant tissue comprising the transgene; isolating the regenerated shoot from the transformed soybean plant tissue comprising the transgene; bringing the isolated regenerated shoot into contact with a membrane, wherein a plant material comprising the transformed soybean plant tissue comprising the transgene is transferred from the isolated regenerated shoot and fixed to the membrane; assaying the transferred plant material to determine a location of the transgene within the transferred plant material; determining a transgene location within the isolated regenerated shoot, wherein the transgene location is selected from the group consisting of a L2/L3 tissue layer and a L1 tissue layer; and identifying an isolated regenerated shoot exhibiting a L2/L3 tissue layer transgene location as a soybean germline transformant.

In a further aspect of this embodiment, a method is provided for regenerating a mature transgenic soybean plant from the identified soybean germline transformant, comprising: selecting the isolated regenerated shoot comprising the soybean germline transformant; and culturing the isolated regenerated shoot comprising the soybean germline transformant into a mature transgenic soybean plant.

In a further aspect, the transforming employs a transformation method elected from the group consisting of Agrobacterium transformation, biolistics, calcium phosphate transformation, polybrene transformation, protoplast fusion transformation, electroporation transformation, ultrasonic transformation, liposome transformation, microinjection transformation, naked DNA transformation, plasmid vector transformation, viral vector transformation, silicon carbide mediated transformation, aerosol beaming transformation, or PEG transformation.

In another aspect, the soybean plant tissue is selected from the group consisting of a meristematic soybean plant tissue, a dermal soybean plant tissue, a ground soybean plant tissue, and a vascular soybean plant tissue.

In another aspect, the meristematic soybean plant tissue comprises apical meristem, primary meristem, or lateral meristem.

In another aspect, the dermal soybean plant tissue comprises epidermis.

In another aspect, the dermal soybean plant tissue comprises periderm.

In another aspect, the vascular soybean plant tissue comprises xylem or phloem.

In another aspect, the transgene is contained within at least one gene expression cassette.

In another aspect, the gene expression cassette comprises a selectable marker gene.

In another aspect, the gene expression cassette comprises a trait gene.

In another aspect, the gene expression cassette comprises an RNAi gene.

In another aspect, the isolating comprises making a horizontal cut transverse to the shoot.

In another aspect, the membrane comprises a nitrocellulose membrane, a nylon membrane, a polytetrafluoroethylene membrane, or a polyvinylidene fluoride membrane.

In another aspect, the assaying comprises a protein detection assay.

In another aspect, the protein detection assay comprises an antibody that binds specifically to a protein expressed from the transgene.

In another aspect, the determining comprises recognizing a dot pattern within the transferred plant material fixed to the membrane.

In another aspect, the determining comprises recognizing a ring pattern within the transferred plant material fixed to the membrane.

In addition to the exemplary aspects and embodiments described above, further aspects and embodiments will become apparent by study of the following descriptions.

### BRIEF DESCRIPTION OF THE FIGURES

FIG. 1 illustrates a plasmid map of pDAB9381.

FIG. 2, Panel a, illustrates a magnified chemiluminescent image of a ring pattern observed from a soybean stem section tissue print. FIG. 2, Panel b, Magnified chemiluminescent image of a dot pattern observed from a soybean stem section tissue print. FIG. 2, Panel c, Epi-white light illumination image of the processed nitrocellulose membrane used to identify the location of the soybean stem section tissue prints (dark spots). FIG. 2, Panel d, Chemiluminescent image of the processed nitrocellulose membrane. The binding of an antibody to PhiYFP resulted in a chemiluminescent signal of the soybean stem section tissue print which was classified as either a “ring” or “dot” image pattern. The distinctly bright chemiluminescent signal in the lower right hand corner is 5 ng of purified PhiYFP protein that was dotted on the membrane as a positive control.

FIG. 3 illustrates the confocal fluorescence microscopic images of soybean stem sections illuminated with excitation/emission parameters specific for the detection of the PhiYFP protein. These images demonstrate three classes for the classification of the soybean shoots: Panel a, No fluorescence; Panel b, fluorescence of the epidermal cell layer only; and, Panel c, fluorescence of the pith and cortical layers.

FIG. 4 is a schematic view of a developing soybean meristem.

### DETAILED DESCRIPTION

### I. Overview

Disclosed herein is a method for screening and detecting soybean germline transformants at the initial stages of the plant transformation process. The disclosed method has been designed for rapid identification and characterization of soybean germline transformants. Briefly, an analysis of transformed soybean plant shoot tissue which comprises a transgene is completed by bringing the shoot tissue into contact with a membrane and assaying the membrane to determine the location of the transgene within the plant material. Soybean germline transformants are identified as those expressing a transgene within the core (L2 and L3) layers of the soybean shoots. A schematic view of a developing soybean meristem is shown in FIG. 4. Identified soybean germline transformants are selected and cultured into mature soybean plants. Other, soybean non-germline transformants are culled from the transformation process at an early stage. As such, soybean plant transformants can be analyzed and screened to identify and select specific transformants which have a donor DNA polynucleotide inserted within the germline tissues.

The disclosed method is a significant improvement over other known methods which have been developed to identify and select soybean germline transformants. See, U.S. Pat. No. 5,503,998, U.S. Pat. No. 5,830,728, and U.S. Pat. No. 5,989,915. These previous screening methods were applicable for particle bombardment transformation and selection using a histochemical assay of the actual stem. The development of a method which can be utilized for screening of soybean transformants generated using any known transformation protocol and which does not require subjecting the transformed soybean tissues to chemical staining is highly desirable as the method can improve the efficiency of the transformation process. Such a method is disclosed in this application.

### II. Terms

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises,” “comprising,” “includes,” “including,” “has,” “having,” “contains,” or “containing,” or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of an embodiment of the disclosure are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as disclosed in the application.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

Isolated: An “isolated” biological component (such as a nucleic acid or polypeptide) has been substantially separated, produced apart from, or purified away from other biological components in the cell of the organism in which the component naturally occurs (i.e., other chromosomal and extra-chromosomal DNA and RNA, and proteins), while effecting a chemical or functional change in the component (e.g., a nucleic acid may be isolated from a chromosome by breaking chemical bonds connecting the nucleic acid to the remaining DNA in the chromosome). Nucleic acid molecules and proteins that have been “isolated” include nucleic acid molecules and proteins purified by standard purification methods. The term also embraces nucleic acids and proteins prepared by recombinant expression in a host cell, as well as chemically-synthesized nucleic acid molecules, proteins, and peptides.

Nucleic acid: The terms “polynucleotide,” “nucleic acid,” and “nucleic acid molecule” are used interchangeably herein, and encompass a singular nucleic acid; plural nucleic acids; a nucleic acid fragment, variant, or derivative thereof; and nucleic acid construct (e.g., messenger RNA (mRNA) and plasmid DNA (pDNA)). A polynucleotide or nucleic acid may contain the nucleotide sequence of a full-length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single- and double-stranded regions; single- and double-stranded RNA; and RNA that is mixture of single- and double-stranded regions. Hybrid molecules comprising DNA and RNA may be single-stranded, double-stranded, or a mixture of single- and double-stranded regions. The foregoing terms also include chemically, enzymatically, and metabolically modified forms of a polynucleotide or nucleic acid.

It is understood that a specific DNA refers also to the complement thereof, the sequence of which is determined according to the rules of deoxyribonucleotide base-pairing.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product.

As used herein, the term “coding sequence” refers to a nucleic acid sequence that encodes a specific amino acid sequence. A “regulatory sequence” refers to a nucleotide sequence located upstream (e.g., 5′ non-coding sequences), within, or downstream (e.g., 3′ non-coding sequences) of a coding sequence, which influence the transcription, RNA processing or stability, or translation of the associated coding sequence. Regulatory sequences include, for example and without limitation: promoters; translation leader sequences; introns; polyadenylation recognition sequences; RNA processing sites; effector binding sites; and stem-loop structures.

Hybridization: A nucleic acid comprising all or part of a nucleotide sequence may be used as a probe that selectively “hybridizes” to nucleotide sequences present in a population of cloned genomic DNA fragments or cDNA fragments (e.g., genomic or cDNA libraries from a chosen organism) that have a significant amount of sequence identity to the probe sequence. A hybridization probe may be a genomic DNA fragment; a plasmid DNA fragment; a cDNA fragment; an RNA fragment; a PCR amplified DNA fragment; an oligonucleotide; or other polynucleotide, and a probe may be labeled with a detectable group (e.g., ³²P), or any other detectable marker. Thus, for example and without limitation, a probe for hybridization may be made by labeling a synthetic oligonucleotide that specifically hybridizes to a nucleic acid herein (e.g., a nucleic acid having at least about 90% identity to SEQ ID NO:1). Methods for preparation of probes for hybridization, and for construction of cDNA and genomic libraries, are known in the art. Sambrook et al. (1989), Molecular Cloning: A Laboratory Manual, Second Edition, Cold Spring Harbor Laboratory Press, Cold Spring Harbor, N.Y. An extensive guide to the hybridization of nucleic acids can be found in Sambrook et al. (1989), supra; and Ausubel et al. (1997), Short Protocols in Molecular Biology, Third Edition, Wiley, NY, New York, pp. 2-40.

As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of “polypeptide,” and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis.

Endogenous and Heterologous: As used herein, the term “native” refers to the form of a polynucleotide, gene or polypeptide that is found in nature with its own regulatory sequences, if present. The term “endogenous” refers to the native form of the polynucleotide, gene or polypeptide in its natural location in the organism or in the genome of the organism.

In contrast, the term “heterologous” refers to a polynucleotide, gene or polypeptide that is not normally found at its location in the reference (host) organism. For example, a heterologous nucleic acid may be a nucleic acid that is normally found in the reference organism at a different genomic location. By way of further example, a heterologous nucleic acid may be a nucleic acid that is not normally found in the reference organism. A host organism comprising a heterologous polynucleotide, gene or polypeptide may be produced by introducing the heterologous polynucleotide, gene or polypeptide into the host organism. In particular examples, a heterologous polynucleotide comprises a native coding sequence, or portion thereof, that is reintroduced into a source organism in a form that is different from the corresponding native polynucleotide. In particular examples, a heterologous gene comprises a native coding sequence, or portion thereof, that is reintroduced into a source organism in a form that is different from the corresponding native gene. For example, a heterologous gene may include a native coding sequence that is a portion of a chimeric gene including non-native regulatory regions that is reintroduced into the native host. In particular examples, a heterologous polypeptide is a native polypeptide that is reintroduced into a source organism in a form that is different from the corresponding native polypeptide.

A heterologous gene or polypeptide may be a gene or polypeptide that comprises a functional polypeptide or nucleic acid sequence encoding a functional polypeptide that is fused to another genes or polypeptide to produce a chimeric or fusion polypeptide, or a gene encoding the same. Genes and proteins of particular embodiments include specifically exemplified full-length sequences and portions, segments, fragments (including contiguous fragments and internal and/or terminal deletions compared to the full-length molecules), variants, mutants, chimerics, and fusions of these sequences.

Modification: As used herein, the term “modification” may refer to a change in a particular reference polynucleotide that results in reduced, substantially eliminated, or eliminated activity of a polypeptide encoded by the reference polynucleotide. A modification may also refer to a change in a reference polypeptide that results in reduced, substantially eliminated, or eliminated activity of the reference polypeptide. Alternatively, the term “modification” may refer to a change in a reference polynucleotide that results in increased or enhanced activity of a polypeptide encoded by the reference polynucleotide, as well as a change in a reference polypeptide that results in increased or enhanced activity of the reference polypeptide. Changes such as the foregoing may be made by any of several methods well-known in the art including, for example and without limitation: deleting a portion of the reference molecule; mutating the reference molecule (e.g., via spontaneous mutagenesis, via random mutagenesis, via mutagenesis caused by mutator genes, and via transposon mutagenesis); substituting a portion of the reference molecule; inserting an element into the reference molecule; down-regulating expression of the reference molecule; altering the cellular location of the reference molecule; altering the state of the reference molecule (e.g., via methylation of a reference polynucleotide, and via phosphorylation or ubiquitination of a reference polypeptide); removing a cofactor of the reference molecule; introduction of an antisense RNA/DNA targeting the reference molecule; introduction of an interfering RNA/DNA targeting the reference molecule; chemical modification of the reference molecule; covalent modification of the reference molecule; irradiation of the reference molecule with UV radiation or X-rays; homologous recombination that alters the reference molecule; mitotic recombination that alters the reference molecule; replacement of the promoter of the reference molecule; and/or combinations of any of the foregoing.

Guidance in determining which nucleotides or amino acid residues may be modified in a specific example may be found by comparing the sequence of the reference polynucleotide or polypeptide with that of homologous (e.g., homologous yeast or bacterial) polynucleotides or polypeptides, and maximizing the number of modifications made in regions of high homology (conserved regions) or consensus sequences.

Promoter: The term “promoter” refers to a DNA sequence capable of controlling the expression of a nucleic acid coding sequence or functional RNA. In examples, the controlled coding sequence is located 3′ to a promoter sequence. A promoter may be derived in its entirety from a native gene, a promoter may be comprised of different elements derived from different promoters found in nature, or a promoter may even comprise synthetic DNA segments. It is understood by those skilled in the art that different promoters can direct the expression of a gene in different tissues or cell types, or at different stages of development, or in response to different environmental or physiological conditions. Examples of all of the foregoing promoters are known and used in the art to control the expression of heterologous nucleic acids. Promoters that direct the expression of a gene in most cell types at most times are commonly referred to as “constitutive promoters.” Furthermore, while those in the art have (in many cases unsuccessfully) attempted to delineate the exact boundaries of regulatory sequences, it has come to be understood that DNA fragments of different lengths may have identical promoter activity. The promoter activity of a particular nucleic acid may be assayed using techniques familiar to those in the art.

Operably linked: The term “operably linked” refers to an association of nucleic acid sequences on a single nucleic acid, wherein the function of one of the nucleic acid sequences is affected by another. For example, a promoter is operably linked with a coding sequence when the promoter is capable of effecting the expression of that coding sequence (e.g., the coding sequence is under the transcriptional control of the promoter). A coding sequence may be operably linked to a regulatory sequence in a sense or antisense orientation.

Expression: The term “expression,” as used herein, may refer to the transcription and stable accumulation of sense (mRNA) or antisense RNA derived from a DNA. Expression may also refer to translation of mRNA into a polypeptide. As used herein, the term “overexpression” refers to expression that is higher than endogenous expression of the same gene or a related gene. Thus, a heterologous gene is “overexpressed” if its expression is higher than that of a comparable endogenous gene.

Transformation: As used herein, the term “transformation” refers to the transfer and integration of a nucleic acid or fragment thereof into a host organism, resulting in genetically stable inheritance. Host organisms containing a transforming nucleic acid are referred to as “transgenic,” “recombinant,” or “transformed” organisms. Known methods of transformation include, for example: Agrobacterium tumefaciens or A. rhizogenes-mediated transformation; calcium phosphate transformation; polybrene transformation; protoplast fusion; electroporation; ultrasonic methods (e.g., sonoporation); liposome transformation; microinjection; transformation with naked DNA; transformation with plasmid vectors; transformation with viral vectors; biolistic transformation (microparticle bombardment); silicon carbide WHISKERS-mediated transformation; aerosol beaming; and PEG-mediated transformation.

Introduced: As used herein, the term “introduced” (in the context of introducing a nucleic acid into a cell) includes transformation of a cell, as well as crossing a plant comprising the nucleic acid with a second plant, such that the second plant contains the nucleic acid, as may be performed utilizing conventional plant breeding techniques. Such breeding techniques are known in the art. For a discussion of plant breeding techniques, see Poehlman (1995), Breeding Field Crops, 4^{th} Edition, AVI Publication Co., Westport Conn.

Backcrossing methods may be used to introduce a nucleic acid into a plant. This technique has been used for decades to introduce traits into plants. An example of a description of backcrossing (and other plant breeding methodologies) can be found in, for example, Poelman (1995), supra; and Jensen (1988), Plant Breeding Methodology, Wiley, New York, N.Y. In an exemplary backcross protocol, an original plant of interest (the “recurrent parent”) is crossed to a second plant (the “non-recurrent parent”) that carries the a nucleic acid be introduced. The resulting progeny from this cross are then crossed again to the recurrent parent, and the process is repeated until a converted plant is obtained, wherein essentially all of the desired morphological and physiological characteristics of the recurrent parent are recovered in the converted plant, in addition to the nucleic acid from the non-recurrent parent.

Plasmid/vector: The terms “plasmid” and “vector,” as used herein, refer to an extra chromosomal element that may carry one or more gene(s) that are not part of the central metabolism of the cell. Plasmids and vectors typically are circular double-stranded DNA molecules. However, plasmids and vectors may be linear or circular nucleic acids, of a single- or double-stranded DNA or RNA, and may be derived from any source, in which a number of nucleotide sequences have been joined or recombined into a unique construction that is capable of introducing a promoter fragment and a coding DNA sequence along with any appropriate 3′ untranslated sequence into a cell. In examples, plasmids and vectors may comprise autonomously replicating sequences, genome integrating sequences, and/or phage or nucleotide sequences.

Polypeptide and “protein” are used interchangeably herein and include a molecular chain of two or more amino acids linked through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included, within the meaning of polypeptide. The terms also include molecules in which one or more amino acid analogs or non-canonical or unnatural amino acids are included as can be synthesized, or expressed recombinantly using known protein engineering techniques. In addition, inventive fusion proteins can be derivatized as described herein by well-known organic chemistry techniques.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

### III. Soybean Tissues and Parts

In some embodiments, a soybean plant tissue is provided that comprises a transgene. Some embodiments include a soybean plant tissue comprising a shoot or a plant material transferred from the shoot. In additional embodiments the soybean plant tissue comprises a core or mantle tissue. In further embodiments the soybean plant tissue is selected from the group consisting of a meristematic soybean plant tissue, a dermal soybean plant tissue, a ground soybean plant tissue, and a vascular soybean plant tissue.

A plant cell, plant part, and/or plant may be transformed with a transgene to comprise a heterologous polypeptide and/or heterologous nucleic acid by any of several transformation methods known in the art. In particular embodiments herein, a transgene is introduced into a plant cell, plant part, and/or plant by a method selected from, for example and without limitation: transformation and selective breeding (e.g., backcross breeding).

The plant cells form plant tissues which can be categorized as meristematic tissue, dermal tissue, ground tissue, or vascular tissue. In an embodiment the soybean plant tissue which is transformed with a transgene can comprise a meristematic soybean plant tissue, a dermal plant tissue, a ground plant tissue or a vascular soybean plant tissue. Use of a transgene for transformation of core tissues (L2 and L3 layers) which comprise the meristematic and vascular plant tissues results in the transformation of a soybean germline tissue. Use of a transgene for transformation of mantle tissues (L1 layer) which comprise the ground and dermal plant tissues results in the transformation of a soybean non-germline tissue.

The meristematic tissue comprises apical meristem, primary meristem, or lateral meristem. These undifferentiated tissues undergo division of new cells which are used for growth or repair of the plant tissues, and are characterized as zones of actively dividing cells. Cell division occurs solely in the meristematic tissues. Apical meristems which are located at the shoot tips are directly involved in shoot elongation. Lateral meristems, such as the vascular meristem are involved in internal growth, these cells surround the established stem of a plant and cause it to grow laterally.

The vascular tissue are a mixture of differentiated cells consisting of parenchyma cells, sclerenchyma cells, fiber cells, and other cells involved in transport (i.e., vessels, tracheids, xylem, or phloem). These types of cells transport fluids such as water and nutrients internally within the plant cell.

The dermal and ground tissue are non-meristematic tissues (non-dividing tissue) which are made up of parenchyma cells, sclerenchyma cells, and collenchyma cells. The dermal tissues comprise the outermost cell layers of the plants leaves, roots, stems, fruits or seeds. The ground tissues are simple, non-meristematic tissues made up of parenchyma cells, sclerenchyma cells, chlorenchyma, and collenchyma cells. These cell types generally form the pith and cortex of the stems.

In an embodiment, the subject disclosure describes a method for identifying a soybean germline transformant which comprises a transgene. In a preferred embodiment, a soybean plant tissue is transformed via an Agrobacterium-mediated method of modified half-seed explants (M. Paz, et al. (2005), Plant Cell Rep., 25: 206-213) or via a cotyledonary node transformation method (P. Zeng, et al. (2004), Plant Cell Rep., 22(7): 478-482). Using either method, the transgene is delivered to soybean plant tissues which comprise the outer mantle tissue (L1 layer) or delivered to underlying tissues located deeper within the plant, such as the core tissues (L2 and L3 layers). The mantle tissue (L1 layer) will divide to form the epidermal and ground tissues which comprise non-germline cells. The core tissues divide to form the meristematic and vascular tissues which comprise germline cells. Only the transgenic events with transformed germline cells can pass the transgene to the next generation. The median vertical section of a soybean shoot apical meristem is illustrated as FIG. 4 to show the organization of the L1, L2 and L3 layers of the soybean shoot.

Any soybean plant cell may be genetically modified to comprise a transgene. In some embodiments, the plant cell that is so genetically modified is not capable of regeneration to produce a plant (i.e., non-germline transformant). In some embodiments, plants which are genetically modified in accordance with the present disclosure are capable of regeneration to produce a transgenic plant (i.e., germline transformant).

Nucleic acids introduced into a soybean plant cell may be used to confer desired agronomic traits in soybean. A wide variety of soybean plants and plant cell systems may be engineered for the desired physiological and agronomic characteristics described herein using a nucleic acid and various transformation methods. Embodiments herein may use any of many methods for the transformation of plants (and production of genetically modified plants) that are known in the art. Numerous methods for plant transformation have been developed, including biological and physical transformation protocols for dicotyledenous plants, as well as monocotyledenous plants (see, e.g., Goto-Fumiyuki et al. (1999), Nat. Biotechnol. 17:282-6; Mild et al. (1993), Methods in Plant Molecular Biology and Biotechnology (B. R. Glick and J. E. Thompson, Eds.), CRC Press, Inc., Boca Raton, Fla., pp. 67-88). In addition, vectors and in vitro culture methods for plant cell and tissue transformation and regeneration of plants are described, for example, in Gruber et al. (1993), supra, at pp. 89-119.

Plant transformation methodologies available for introducing a nucleic acid into a plant host cell include, for example and without limitation: transformation with disarmed T-DNA using Agrobacterium tumefaciens or A. rhizogenes as the transformation agent; calcium phosphate transfection; polybrene transformation; protoplast fusion; electroporation (D'Halluin et al. (1992), Plant Cell 4:1495-505); ultrasonic methods (e.g., sonoporation); liposome transformation; microinjection; contact with naked DNA; contact with plasmid vectors; contact with viral vectors; biolistics (e.g., DNA particle bombardment (see, e.g., Klein et al. (1987), Nature 327:70-3) and microparticle bombardment (Sanford et al. (1987), Part. Sci. Technol. 5:27; Sanford (1988), Trends Biotech. 6:299, Sanford (1990), Physiol. Plant 79:206; and Klein et al. (1992), Biotechnology 10:268); silicon carbide WHISKERS™-mediated transformation (Kaeppler et al. (1990), Plant Cell Rep. 9:415-8); nanoparticle transformation (see, e.g., U.S. Patent Publication No. US2009/0104700A1); aerosol beaming; and polyethylene glycol (PEG)-mediated uptake. In specific examples, a transgene may be introduced directly into the genomic DNA of a soybean plant cell via one of the previously described transformation protocols.

A widely utilized method for introducing a gene expression cassette comprising a transgene into a plant is based on the natural transformation system of Agrobacterium. Horsch et al. (1985), Science 227:1229. A. tumefaciens and A. rhizogenes are plant pathogenic soil bacteria known to be useful to genetically transform plant cells. The Ti and Ri plasmids of A. tumefaciens and A. rhizogenes, respectively, carry genes responsible for genetic transformation of the plant. Kado (1991), Crit. Rev. Plant. Sci. 10:1. Details regarding Agrobacterium vector systems and methods for Agrobacterium-mediated gene transfer are also available in, for example, Gruber et al., supra, Mild et al., supra, Moloney et al. (1989), Plant Cell Reports 8:238, and U.S. Pat. Nos. 4,940,838 and 5,464,763.

If Agrobacterium is used for the transformation, the DNA to be inserted typically is cloned into special plasmids, either in an intermediate vector or a binary vector. Intermediate vectors cannot replicate themselves in Agrobacterium. The intermediate vector may be transferred into A. tumefaciens by means of a helper plasmid (conjugation). The Japan Tobacco Superbinary system is an example of such a system (reviewed by Komori et al. (2006), Methods in Molecular Biology (K. Wang, ed.) No. 343; Agrobacterium Protocols, 2^{nd} Edition, Vol. 1, Humana Press Inc., Totowa, N.J., pp. 15-41; and Komori et al. (2007), Plant Physiol. 145:1155-60). Binary vectors can replicate themselves both in E. coli and in Agrobacterium. Binary vectors comprise a selection marker gene and a linker or polylinker which are framed by the right and left T-DNA border regions. They can be transformed directly into Agrobacterium (Holsters, 1978). The Agrobacterium comprises a plasmid carrying a vir region. The Ti or Ri plasmid also comprises the vir region necessary for the transfer of the T-DNA. The vir region is necessary for the transfer of the T-DNA into the plant cell. Additional T-DNA may be contained.

The virulence functions of the Agrobacterium tumefaciens host will direct the insertion of a T-strand containing the gene expression cassette and adjacent marker into the plant cell DNA when the cell is infected by the bacteria using a binary T DNA vector (Bevan (1984), Nuc. Acid Res. 12:8711-21) or the co-cultivation procedure (Horsch et al. (1985), Science 227:1229-31). Generally, the Agrobacterium transformation system is used to engineer dicotyledonous plants. Bevan et al. (1982), Ann. Rev. Genet. 16:357-84; Rogers et al. (1986), Methods Enzymol. 118:627-41. The Agrobacterium transformation system may also be used to transform, as well as transfer, nucleic acids to monocotyledonous plants and plant cells. See U.S. Pat. No. 5,591,616; Hernalsteen et al. (1984), EMBO J. 3:3039-41; Hooykass-Van Slogteren et al. (1984), Nature 311:763-4; Grimsley et al. (1987), Nature 325:1677-9; Boulton et al. (1989), Plant Mol. Biol. 12:31-40; and Gould et al. (1991), Plant Physiol. 95:426-34.

The genetic manipulations of a recombinant host herein may be performed using standard recombinant DNA techniques and screening, and may be carried out in any host cell that is suitable to genetic manipulation. In some embodiments, a recombinant host cell may be any soybean plant or variety suitable for genetic modification and/or recombinant gene expression. In some embodiments, a recombinant host may be a soybean germline transformant plant. Standard recombinant DNA and molecular cloning techniques used here are well-known in the art and are described in, for example and without limitation: Sambrook et al. (1989), supra; Silhavy et al. (1984), Experiments with Gene Fusions, Cold Spring Harbor Laboratory Press, Cold Spring Harbor, N.Y.; and Ausubel et al. (1987), Current Protocols in Molecular Biology, Greene Publishing Assoc. and Wiley-Interscience, New York, N.Y.

The transformation methods result in transgenic plants. The subject disclosure can be utilized to identify specific transgenic plants which comprise soybean germline transformants, particularly transformants derived from the core tissue (L2 and L3). In particular, the soybean germline transformants produced by transformation methods provided by the invention can be transmitted to subsequent generations.

Following the introduction of a transgene into a soybean plant cell, the plant cell may be grown, and upon emergence of differentiating tissue such as shoots and roots, mature plants can be generated. In some embodiments, a plurality of soybean plants can be generated. Methodologies for regenerating plants are known to those of ordinary skill in the art and can be found, for example, in: Plant Cell and Tissue Culture, 1994, Vasil and Thorpe Eds. Kluwer Academic Publishers, and in: Plant Cell Culture Protocols (Methods in Molecular Biology 111, 1999 Hall Eds Humana Press). Genetically modified soybean plants described herein may be cultured in a fermentation medium or grown in a suitable medium such as soil. In some embodiments, a suitable growth medium for higher plants may be any growth medium for plants, including, but not limited to, soil, sand, any other particulate media that support root, growth (e.g., perlite, etc.) or hydroponic culture, as well as suitable light, water and nutritional supplements that facilitate the growth of the higher plant.

Transformed soybean plant cells which are produced by any of the above transformation techniques can be cultured to regenerate a mature soybean plant that possesses the transformed genotype, and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans, et al., “Protoplasts Isolation and Culture” in Handbook of Plant Cell Culture, pp. 124-176, Macmillian Publishing Company, New York, 1983; and Binding, Regeneration of Plants, Plant Protoplasts, pp. 21-73, CRC Press, Boca Raton, 1985. Regeneration can also be obtained from plant callus, explants, organs, pollens, embryos or parts thereof. Such regeneration techniques are described generally in Klee et al. (1987), Ann. Rev. of Plant Phys. 38:467-486.

In other embodiments, the soybean plant cells which are transformed are not capable of regeneration to produce a mature soybean plant. Such transformed soybean plant cells are non-germline soybean transformants.

A transformed soybean plant cell, callus, tissue or plant may be identified and isolated by selecting or screening the engineered plant material for traits encoded by the marker genes present on the transforming DNA. For instance, selection can be performed by growing the engineered plant material on media containing an inhibitory amount of the antibiotic or herbicide to which the transforming gene construct confers resistance. Further, transformed plants and plant cells can also be identified by screening for the activities of any visible marker genes (e.g., the β-glucuronidase, luciferase, or gfp genes) that may be present on the recombinant nucleic acid constructs. Such selection and screening methodologies are well known to those skilled in the art.

A transgenic soybean plant containing a heterologous molecule herein can be produced through selective breeding, for example, by sexually crossing a first parental plant comprising the molecule, and a second parental plant, thereby producing a plurality of first progeny plants. A first progeny plant may then be selected that is resistant to a selectable marker (e.g., glyphosate, resistance to which may be conferred upon the progeny plant by the heterologous molecule herein). The first progeny plant may then be selfed, thereby producing a plurality of second progeny plants. Then, a second progeny plant may be selected that is resistant to the selectable marker. These steps can further include the back-crossing of the first progeny plant or the second progeny plant to the second parental plant or a third parental plant.

It is also to be understood that two different transgenic soybean plants can also be mated to produce offspring that contain two independently segregating, added, exogenous genes. Selfing of appropriate progeny can produce plants that are homozygous for both added, exogenous genes. Back-crossing to a parental plant and out-crossing with a non-transgenic plant are also contemplated, as is vegetative propagation. Other breeding methods commonly used for different traits and crops are known in the art. Backcross breeding has been used to transfer genes for a simply inherited, highly heritable trait into a desirable homozygous cultivar or inbred line, which is the recurrent parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

A transgene may also be introduced into a predetermined area of the plant genome through homologous recombination. Methods to stably integrate a polynucleotide sequence within a specific chromosomal site of a plant cell via homologous recombination have been described within the art. For instance, site specific integration as described in U.S. Patent Application Publication No. 2009/0111188 A1 involves the use of recombinases or integrases to mediate the introduction of a donor polynucleotide sequence into a chromosomal target. In addition, International Patent Application No. WO 2008/021207, describes zinc finger mediated-homologous recombination to stably integrate one or more donor polynucleotide sequences within specific locations of the genome. The use of recombinases such as FLP/FRT as described in U.S. Pat. No. 6,720,475, or CRE/LOX as described in U.S. Pat. No. 5,658,772, can be utilized to stably integrate a polynucleotide sequence into a specific chromosomal site. Finally, the use of meganucleases for targeting donor polynucleotides into a specific chromosomal location was described in Puchta et al. (1996), PNAS USA 93 pp. 5055-5060).

Other various methods for site specific integration within plant cells are generally known and applicable (Kumar et al. (2001), Trends in Plant Sci. 6(4) pp. 155-159). Furthermore, site-specific recombination systems that have been identified in several prokaryotic and lower eukaryotic organisms may be applied for use in plants. Examples of such systems include, but are not limited too; the R/RS recombinase system from the pSR1 plasmid of the yeast Zygosaccharomyces rouxii (Araki et al. (1985), J. Mol. Biol. 182:191-203), and the Gin/gix system of phage Mu (Maeser and Kahlmann (1991), Mol. Gen. Genet. 230:170-176).

### IV. Agronomic Trait-Encoding Sequences

Some embodiments herein provide a transgene encoding a polypeptide comprising a gene expression cassette. Such a transgene may be useful in any of a wide variety of applications to produce transgenic soybean plants. Particular examples of a transgene comprising a gene expression cassette are provided for illustrative purposes herein and include a gene expression comprising a trait gene, an RNAi gene, or a selectable marker gene.

In engineering a gene for expression in soybean plants, the codon bias of the prospective host plant(s) may be determined, for example, through use of publicly available DNA sequence databases to find information about the codon distribution of plant genomes or the protein coding regions of various plant genes.

In designing coding regions in a nucleic acid for plant expression, the primary (“first choice”) codons preferred by the plant should be determined, as well as the second, third, fourth, etc., choices of preferred codons, when multiple choices exist. A new DNA sequence can then be designed which encodes the amino acid sequence of the same peptide, but the new DNA sequence differs from the original DNA sequence by the substitution of plant (first preferred, second preferred, third preferred, or fourth preferred, etc.) codons to specify the amino acid at each position within the amino acid sequence.

The new sequence may then be analyzed for restriction enzyme sites that might have been created by the modifications. The identified sites may be further modified by replacing the codons with first, second, third, or fourth choice preferred codons. Other sites in the sequence that could affect transcription or translation of the gene of interest are stem-loop structures, exon:intron junctions (5′ or 3′), poly A addition signals, and RNA polymerase termination signals; these sites may be removed by the substitution of plant codons. The sequence may be further analyzed and modified to reduce the frequency of TA or CG doublets. In addition to the doublets, G or C sequence blocks that have more than about six residues that are the same can affect transcription or translation of the sequence. Therefore, these blocks may be modified by replacing the codons of first or second choice, etc., with the next preferred codon of choice.

Once an optimized (e.g., a plant-optimized) DNA sequence has been designed on paper, or in silico, actual DNA molecules may be synthesized in the laboratory to correspond in sequence precisely to the designed sequence. Such synthetic nucleic acid molecule molecules can be cloned and otherwise manipulated exactly as if they were derived from natural or native sources.

A nucleic acid herein may be cloned into a vector for transformation into prokaryotic or eukaryotic cells for replication and/or expression. Vectors may be prokaryotic vectors; e.g., plasmids, or shuttle vectors, insect vectors, or eukaryotic vectors. A nucleic acid herein may also be cloned into an expression vector, for example, for administration to a plant cell. In certain applications, it may be preferable to have vectors that are functional in E. coli (e.g., production of protein for raising antibodies, DNA sequence analysis, construction of inserts, obtaining quantities of nucleic acids).

In an embodiment, a transgene to be expressed is disclosed in the subject application. The gene expression cassette may comprise a selectable marker gene, a trait gene, or an RNAi gene. Examples of a selectable marker gene, a trait gene, and an RNAi gene are further provided below. The methods disclosed in the present application are advantageous in that they provide a method for selecting germline transformants that is not dependent on the specific function of the protein product, or other function, of the transgene.

Transgenes or Coding Sequence that Confer Resistance to Pests or Disease

(A) Plant Disease Resistance Genes. Plant defenses are often activated by specific interaction between the product of a disease resistance gene (R) in the plant and the product of a corresponding avirulence (Avr) gene in the pathogen. A plant variety can be transformed with cloned resistance gene to engineer plants that are resistant to specific pathogen strains. Examples of such genes include, the tomato Cf-9 gene for resistance to Cladosporium fulvum (Jones et al., 1994 Science 266:789), tomato Pto gene, which encodes a protein kinase, for resistance to Pseudomonas syringae pv. tomato (Martin et al., 1993 Science 262:1432), and Arabidopsis RSSP2 gene for resistance to Pseudomonas syringae (Mindrinos et al., 1994 Cell 78:1089).

(B) A Bacillus thuringiensis protein, a derivative thereof or a synthetic polypeptide modeled thereon, such as, a nucleotide sequence of a Bt δ-endotoxin gene (Geiser et al., 1986 Gene 48:109), and a vegetative insecticidal (VIP) gene (see, e.g., Estruch et al. (1996), Proc. Natl. Acad. Sci. 93:5389-94). Moreover, DNA molecules encoding δ-endotoxin genes can be purchased from American Type Culture Collection (Rockville, Md.), under ATCC accession numbers 40098, 67136, 31995 and 31998.

(C) A lectin, such as, nucleotide sequences of several Clivia miniata mannose-binding lectin genes (Van Damme et al., 1994 Plant Molec. Biol. 24:825).

(D) A vitamin binding protein, such as avidin and avidin homologs which are useful as larvicides against insect pests. See U.S. Pat. No. 5,659,026.

(E) An enzyme inhibitor, e.g., a protease inhibitor or an amylase inhibitor. Examples of such genes include a rice cysteine proteinase inhibitor (Abe et al., 1987 J. Biol. Chem. 262:16793), a tobacco proteinase inhibitor I (Huub et al., 1993 Plant Molec. Biol. 21:985), and an α-amylase inhibitor (Sumitani et al., 1993 Biosci. Biotech. Biochem. 57:1243).

(F) An insect-specific hormone or pheromone such as an ecdysteroid and juvenile hormone a variant thereof, a mimetic based thereon, or an antagonist or agonist thereof, such as baculovirus expression of cloned juvenile hormone esterase, an inactivator of juvenile hormone (Hammock et al., 1990 Nature 344:458).

(G) An insect-specific peptide or neuropeptide which, upon expression, disrupts the physiology of the affected pest (J. Biol. Chem. 269:9). Examples of such genes include an insect diuretic hormone receptor (Regan, 1994), an allostatin identified in Diploptera punctata (Pratt, 1989), and insect-specific, paralytic neurotoxins (U.S. Pat. No. 5,266,361).

(H) An insect-specific venom produced in nature by a snake, a wasp, etc., such as a scorpion insectotoxic peptide (Pang, 1992 Gene 116:165).

(I) An enzyme responsible for a hyperaccumulation of monoterpene, a sesquiterpene, a steroid, hydroxamic acid, a phenylpropanoid derivative or another non-protein molecule with insecticidal activity.

(J) An enzyme involved in the modification, including the post-translational modification, of a biologically active molecule; for example, glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. Examples of such genes include, a callas gene (PCT published application WO93/02197), chitinase-encoding sequences (which can be obtained, for example, from the ATCC under accession numbers 3999637 and 67152), tobacco hookworm chitinase (Kramer et al., 1993 Insect Molec. Biol. 23:691), and parsley ubi4-2 polyubiquitin gene (Kawalleck et al., 1993 Plant Molec. Biol. 21:673).

(K) A molecule that stimulates signal transduction. Examples of such molecules include nucleotide sequences for mung bean calmodulin cDNA clones (Botella et al., 1994 Plant Molec. Biol. 24:757) and a nucleotide sequence of a maize calmodulin cDNA clone (Griess et al., 1994 Plant Physiol. 104:1467).

(L) A hydrophobic moment peptide. See U.S. Pat. Nos. 5,659,026 and 5,607,914; the latter teaches synthetic antimicrobial peptides that confer disease resistance.

(M) A membrane permease, a channel former or a channel blocker, such as a cecropin-β lytic peptide analog (Jaynes et al., 1993 Plant Sci. 89:43) which renders transgenic tobacco plants resistant to Pseudomonas solanacearum.

(N) A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. See, for example, Beachy et al. (1990), Ann. Rev. Phytopathol. 28:451.

(O) An insect-specific antibody or an immunotoxin derived therefrom. Thus, an antibody targeted to a critical metabolic function in the insect gut would inactivate an affected enzyme, killing the insect. For example, Taylor et al. (1994), Abstract #497, Seventh Int'l Symposium on Molecular Plant-Microbe Interactions shows enzymatic inactivation in transgenic tobacco via production of single-chain antibody fragments.

(P) A virus-specific antibody. See, for example, Tavladoraki et al. (1993), Nature 266:469, which shows that transgenic plants expressing recombinant antibody genes are protected from virus attack.

(Q) A developmental-arrestive protein produced in nature by a pathogen or a parasite. Thus, fungal endo α-1,4-D polygalacturonases facilitate fungal colonization and plant nutrient release by solubilizing plant cell wall homo-α-1,4-D-galacturonase (Lamb et al. (1992), Bio/Technology 10:1436). The cloning and characterization of a gene which encodes a bean endopolygalacturonase-inhibiting protein is described by Toubart et al. (1992 Plant J. 2:367).

(R) A developmental-arrestive protein produced in nature by a plant, such as the barley ribosome-inactivating gene that provides an increased resistance to fungal disease (Longemann et al. (1992), Bio/Technology 10:3305).

(S) RNA interference, in which an RNA molecule is used to inhibit expression of a target gene. An RNA molecule in one example is partially or fully double stranded, which triggers a silencing response, resulting in cleavage of dsRNA into small interfering RNAs, which are then incorporated into a targeting complex that destroys homologous mRNAs. See, e.g., Fire et al., U.S. Pat. No. 6,506,559; Graham et al. U.S. Pat. No. 6,573,099.

Genes that Confer Resistance to a Herbicide

(A) Genes encoding resistance or tolerance to a herbicide that inhibits the growing point or meristem, such as an imidazalinone, sulfonanilide or sulfonylurea herbicide. Exemplary genes in this category code for a mutant ALS enzyme (Lee et al., 1988 EMBO J. 7:1241), which is also known as AHAS enzyme (Miki et al., 1990 Theor. Appl. Genet. 80:449).

(B) One or more additional genes encoding resistance or tolerance to glyphosate imparted by mutant EPSP synthase and aroA genes, or through metabolic inactivation by genes such as GAT (glyphosate acetyltransferase) or GOX (glyphosate oxidase) and other phosphono compounds such as glufosinate (pat and bar genes; DSM-2), and aryloxyphenoxypropionic acids and cyclohexanediones (ACCase inhibitor encoding genes). See, for example, U.S. Pat. No. 4,940,835, which discloses the nucleotide sequence of a foam of EPSP which can confer glyphosate resistance. A DNA molecule encoding a mutant aroA gene can be obtained under ATCC Accession Number 39256, and the nucleotide sequence of the mutant gene is disclosed in U.S. Pat. No. 4,769,061. European patent application No. 0 333 033 and U.S. Pat. No. 4,975,374 disclose nucleotide sequences of glutamine synthetase genes which confer resistance to herbicides such as L-phosphinothricin. The nucleotide sequence of a phosphinothricinacetyl-transferase gene is provided in European application No. 0 242 246. De Greef et al. (1989), Bio/Technology 7:61, describes the production of transgenic plants that express chimeric bar genes coding for phosphinothricin acetyl transferase activity. Exemplary of genes conferring resistance to aryloxyphenoxypropionic acids and cyclohexanediones, such as sethoxydim and haloxyfop, are the Accl-S1, Accl-S2 and Accl-S3 genes described by Marshall et al. (1992), Theor. Appl. Genet. 83:435.

(C) Genes encoding resistance or tolerance to a herbicide that inhibits photosynthesis, such as a triazine (psbA and gs+ genes) and a benzonitrile (nitrilase gene). Przibilla et al. (1991), Plant Cell 3:169 describe the use of plasmids encoding mutant psbA genes to transform Chlamydomonas. Nucleotide sequences for nitrilase genes are disclosed in U.S. Pat. No. 4,810,648, and DNA molecules containing these genes are available under ATCC accession numbers 53435, 67441 and 67442. Cloning and expression of DNA coding for a glutathione S-transferase is described by Hayes et al. (1992), Biochem. J. 285:173.

(D) Genes encoding resistance or tolerance to a herbicide that bind to hydroxyphenylpyruvate dioxygenases (HPPD), enzymes which catalyze the reaction in which para-hydroxyphenylpyruvate (HPP) is transformed into homogentisate. This includes herbicides such as isoxazoles (EP418175, EP470856, EP487352, EP527036, EP560482, EP682659, U.S. Pat. No. 5,424,276), in particular isoxaflutole, which is a selective herbicide for maize, diketonitriles (EP496630, EP496631), in particular, 2-cyano-3-cyclopropyl-1-(2-SO2CH3-4-CF3 phenyl)propane-1,3-dione and 2-cyano-3-cyclopropyl-1-(2-SO2 CH3-4-2,3Cl2phenyl)propane-1,3-dione, triketones (EP625505, EP625508, U.S. Pat. No. 5,506,195), in particular sulcotrione, and pyrazolinates. A gene that produces an overabundance of HPPD in plants can provide tolerance or resistance to such herbicides, including, for example, genes described in U.S. Pat. Nos. 6,268,549 and 6,245,968 and U.S. Patent Publication No. 20030066102.

(E) Genes encoding resistance or tolerance to phenoxy auxin herbicides, such as 2,4-dichlorophenoxyacetic acid (2,4-D) and which may also confer resistance or tolerance to aryloxyphenoxypropionate (AOPP) herbicides. Examples of such genes include the α-ketoglutarate-dependent dioxygenase enzyme (aad-1) gene, described in U.S. Pat. No. 7,838,733.

(F) Genes encoding resistance or tolerance to phenoxy auxin herbicides, such as 2,4-dichlorophenoxyacetic acid (2,4-D) and which may also confer resistance or tolerance to pyridyloxy auxin herbicides, such as fluroxypyr or triclopyr. Examples of such genes include the α-ketoglutarate-dependent dioxygenase enzyme gene (aad-12), described in WO 2007/053482 A2.

(G) Genes encoding resistance or tolerance to dicamba (see, e.g., U.S. Patent Publication No. 20030135879).

(H) Genes providing resistance or tolerance to herbicides that inhibit protoporphyrinogen oxidase (PPO) (see U.S. Pat. No. 5,767,373).

(I) Genes providing resistance or tolerance to triazine herbicides (such as atrazine) and urea derivatives (such as diuron) herbicides which bind to core proteins of photosystem II reaction centers (PS II) (see Brussian et al. (1989), EMBO J. 1989, 8(4):1237-1245).

Genes that Confer or Contribute to a Value-Added Trait

(A) Modified fatty acid metabolism, for example, by transforming maize or Brassica with an antisense gene or stearoyl-ACP desaturase to increase stearic acid content of the plant (Knultzon et al. (1992), Proc. Nat. Acad. Sci. U.S.A. 89:2624).

(B) Decreased phytate content
(1) Introduction of a phytase-encoding gene, such as the Aspergillus niger phytase gene (Van Hartingsveldt et al., 1993 Gene 127:87), enhances breakdown of phytate, adding more free phosphate to the transformed plant.
   (2) A gene could be introduced that reduces phytate content. In maize, this, for example, could be accomplished by cloning and then reintroducing DNA associated with the single allele which is responsible for maize mutants characterized by low levels of phytic acid (Raboy et al., 1990 Maydica 35:383).

(C) Modified carbohydrate composition effected, for example, by transforming plants with a gene coding for an enzyme that alters the branching pattern of starch. Examples of such enzymes include, Streptococcus mucus fructosyltransferase gene (Shiroza et al. (1988), J. Bacteriol. 170:810), Bacillus subtilis levansucrase gene (Steinmetz et al. (1985), Mol. Gen. Genel. 200:220), Bacillus licheniformis α-amylase (Pen et al. (1992), Bio/Technology 10:292), tomato invertase genes (Elliot et al., 1993), barley amylase gene (Sogaard et al. (1993), J. Biol. Chem. 268:22480), and maize endosperm starch branching enzyme II (Fisher et al. (1993), Plant Physiol. 102:10450).

To express a selectable marker gene, a trait gene, or an RNAi gene in a soybean cell, a nucleic acid encoding the protein is typically subcloned into an expression vector that contains a promoter to direct transcription. Suitable bacterial and eukaryotic promoters are well known in the art and described, e.g., in Sambrook et al., Molecular Cloning, A Laboratory Manual (2nd ed. 1989; 3^{rd} ed., 2001); Kriegler, Gene Transfer and Expression: A Laboratory Manual (1990); and Current Protocols in Molecular Biology (Ausubel et al., supra.). Bacterial expression systems for expressing a nucleic acid herein are available in, for example, E. coli, Bacillus sp., and Salmonella (Palva et al., Gene 22:229-235 (1983)). Kits for such expression systems are commercially available. Eukaryotic expression systems for mammalian cells, yeast, and insect cells are well known by those of skill in the art and are also commercially available.

The particular expression vector used to transport the genetic information into the cell is selected with regard to the intended use (e.g., expression in plants, animals, bacteria, fungus, and protozoa). Standard bacterial and animal expression vectors are known in the art and are described in detail, for example, U.S. Patent Publication 20050064474A1 and International Patent Publications WO 05/084190, WO 05/014791 and WO 03/080809. Standard transfection methods can be used to produce bacterial cell lines that express large quantities of protein, which can then be purified using standard techniques.

The selection of a promoter used to direct expression of a nucleic acid herein depends on the particular application. A number of promoters that direct expression of a gene in a plant may be employed in embodiments herein. Such promoters can be selected from constitutive, chemically-regulated, inducible, tissue-specific, and seed-preferred promoters. For example, a strong constitutive promoter suited to the host cell may be used for expression and purification of the expressed proteins. Non-limiting examples of plant promoters include promoter sequences derived from A. thaliana ubiquitin-10 (ubi-10) (Callis, et al., 1990, J. Biol. Chem., 265:12486-12493); A. tumefaciens mannopine synthase (Δmas) (Petolino et al., U.S. Pat. No. 6,730,824); and/or Cassava Vein Mosaic Virus (CsVMV) (Verdaguer et al., 1996, Plant Molecular Biology 31:1129-1139).

Constitutive promoters include, for example, the core Cauliflower Mosaic Virus 35S promoter (Odell et al. (1985), Nature 313:810-812); Rice Actin promoter (McElroy et al. (1990), Plant Cell 2:163-171); Maize Ubiquitin promoter (U.S. Pat. No. 5,510,474; Christensen et al. (1989), Plant Mol. Biol. 12:619-632 and Christensen et al. (1992), Plant Mol. Biol. 18:675-689); pEMU promoter (Last et al. (1991), Theor. Appl. Genet. 81:581-588); ALS promoter (U.S. Pat. No. 5,659,026); Maize Histone promoter (Chabouté et al. Plant Molecular Biology, 8:179-191 (1987)), and the like.

The range of available plant compatible promoters includes tissue specific and inducible promoters. An inducible regulatory element is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory element to activate transcription is present in an inactive form, which is then directly or indirectly converted to the active form by the inducer. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. Typically, the protein factor that binds specifically to an inducible regulatory element to activate transcription is present in an inactive form which is then directly or indirectly converted to the active form by the inducer. A plant cell containing an inducible regulatory element may be exposed to an inducer by externally applying the inducer to the cell or plant such as by spraying, watering, heating or similar methods.

Any inducible promoter can be used in embodiments herein. See Ward et al. Plant Mol. Biol. 22:361-366 (1993). Inducible promoters include, for example and without limitation: ecdysone receptor promoters (U.S. Pat. No. 6,504,082); promoters from the ACE1 system which respond to copper (Mett et al. PNAS 90:4567-4571 (1993)); In2-1 and In2-2 gene from maize which respond to benzenesulfonamide herbicide safeners (U.S. Pat. No. 5,364,780; Hershey et al., Mol. Gen. Genetics 227: 229-237 (1991); and Gatz et al., Mol. Gen. Genetics 243: 32-38 (1994)); Tet repressor from Tn10 (Gatz et al., Mol. Gen. Genet. 227:229-237 (1991)); promoters from a steroid hormone gene, the transcriptional activity of which is induced by a glucocorticosteroid hormone, Schena et al., Proc. Natl. Acad. Sci. U.S.A. 88:10421 (1991); and McNellis et al. (1998), Plant J. 14(2):247-257; the maize GST promoter, which is activated by hydrophobic electrophilic compounds that are used as pre-emergent herbicides (see U.S. Pat. No. 5,965,387 and International Patent Application, Publication No. WO 93/001294); and the tobacco PR-1a promoter, which is activated by salicylic acid (see S. Ono, M. Kusama, R. Ogura, K. Hiratsuka, “Evaluation of the Use of the Tobacco PR-1a Promoter to Monitor Defense Gene Expression by the Luciferase Bioluminescence Reporter System,” Biosci. Biotechnol. Biochem. 2011 Sep. 23; 75(9):1796-800). Other chemical-regulated promoters of interest include tetracycline-inducible and tetracycline-repressible promoters (see, for example, Gatz et al. (1991), Mol. Gen. Genet. 227:229-237, and U.S. Pat. Nos. 5,814,618 and 5,789,156).

Other regulatable promoters of interest include a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84(19):6624-6628 (1987)), inducible by anaerobic conditions; the light-inducible promoter derived from the pea rbcS gene or pea psaDb gene (Yamamoto et al. (1997), Plant J. 12(2):255-265); a light-inducible regulatory element (Feinbaum et al., Mol. Gen. Genet. 226:449, 1991; Lam and Chua, Science 248:471, 1990; Matsuoka et al. (1993), Proc. Natl. Acad. Sci. U.S.A. 90(20):9586-9590; Orozco et al. (1993), Plant Mol. Bio. 23(6):1129-1138); a plant hormone inducible regulatory element (Yamaguchi-Shinozaki et al., Plant Mol. Biol. 15:905, 1990; Kares et al., Plant Mol. Biol. 15:225, 1990), and the like. An inducible regulatory element also can be the promoter of the maize In2-1 or In2-2 gene, which responds to benzenesulfonamide herbicide safeners (Hershey et al., Mol. Gen. Gene, 227:229-237, 1991; Gatz et al., Mol. Gen. Genet. 243:32-38, 1994), and the Tet repressor of transposon Tn10 (Gatz et al., Mol. Gen. Genet. 227:229-237, 1991).

Stress inducible promoters include salt/water stress-inducible promoters such as P5CS (Zang et al. (1997), Plant Sciences 129:81-89); cold-inducible promoters, such as cor15a (Hajela et al. (1990), Plant Physiol. 93:1246-1252), cor15b (Wilhelm et al. (1993), Plant Mol. Biol. 23:1073-1077), wsc120 (Ouellet et al. (1998), FEBS Lett. 423-324-328), ci7 (Kirch et al. (1997), Plant Mol. Biol. 33:897-909), and ci21A (Schneider et al. (1997), Plant Physiol. 113:335-45); drought-inducible promoters, such as Trg-31 (Chaudhary et al. (1996), Plant Mol. Biol. 30:1247-57) and rd29 (Kasuga et al. (1999), Nature Biotechnology 18:287-291); osmotic inducible promoters, such as Rab17 (Vilardell et al. (1991), Plant Mol. Biol. 17:985-93) and osmotin (Raghothama et al. (1993), Plant Mol. Biol. 23:1117-28); heat inducible promoters, such as heat shock proteins (Barros et al. (1992), Plant Mol. 19:665-75; Marrs et al. (1993), Dev. Genet. 14:27-41), smHSP (Waters et al. (1996), J. Experimental Botany 47:325-338); and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993), Mol. Gen. Genetics 236:331-340). Certain promoters are inducible by wounding, including the Agrobacterium pMAS promoter (Guevara-Garcia et al. (1993), Plant J. 4(3):495-505) and the Agrobacterium ORF13 promoter (Hansen et al. (1997), Mol. Gen. Genet. 254(3):337-343).

Tissue-preferred promoters may be utilized to target enhanced transcription and/or expression within a particular plant tissue. Examples of these types of promoters include seed-preferred expression, such as that provided by the phaseolin promoter (Bustos et al. 1989, The Plant Cell Vol. 1, 839-853), and the maize globulin-1 gene, Belanger, et al. 1991 Genetics 129:863-972. For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa zein, γ-zein, waxy, shrunken 1, shrunken 2, globulin 1, etc. Seed-preferred promoters also include those promoters that direct gene expression predominantly to specific tissues within the seed such as, for example, the endosperm-preferred promoter of γ-zein, the cryptic promoter from tobacco (Fobert et al. 1994, T-DNA tagging of a seed coat-specific cryptic promoter in tobacco, Plant J. 4:567-577), the P-gene promoter from corn (Chopra et al. 1996, Alleles of the maize P gene with distinct tissue specificities encode Myb-homologous proteins with C-teiminal replacements, Plant Cell 7:1149-1158, Erratum in Plant Cell, 1997, 1:109), the globulin-1 promoter from corn (Belenger and Kriz, 1991, Molecular basis for Allelic Polymorphism of the maize Globulin-1 gene, Genetics 129:863-972), and promoters that direct expression to the seed coat or hull of corn kernels, for example the pericarp-specific glutamine synthetase promoter (Muhitch et al., 2002, Isolation of a Promoter Sequence From the Glutamine Synthetase₁₋₂ Gene Capable of Conferring Tissue-Specific Gene Expression in Transgenic Maize, Plant Science 163:865-872).

In addition to the promoter, an expression vector typically contains a transcription unit or expression cassette that contains all the additional elements required for the expression of the nucleic acid in host cells, either prokaryotic or eukaryotic. A typical expression cassette thus contains a promoter operably-linked, e.g., to a nucleic acid sequence encoding the protein, and signals required, e.g., for efficient polyadenylation of the transcript, transcriptional termination, ribosome binding sites, or translation termination. Additional elements of the cassette may include, e.g., enhancers and heterologous splicing signals.

Other components of the vector may be included, also depending upon intended use of the gene. Examples include selectable markers, targeting or regulatory sequences, transit peptide sequences such as the optimized transit peptide sequence (see U.S. Pat. No. 5,510,471) stabilizing sequences such as RB7 MAR (see Thompson and Myatt (1997), Plant Mol. Biol., 34: 687-692 and WO9727207) or leader sequences, introns etc. General descriptions and examples of plant expression vectors and reporter genes can be found in Gruber, et al., “Vectors for Plant Transformation” in Methods in Plant Molecular Biology and Biotechnology, Glick et al. eds; CRC Press pp. 89-119 (1993).

The selection of an appropriate expression vector will depend upon the host and the method of introducing the expression vector into the host. The expression cassette may include, at the 3′ terminus of a heterologous nucleotide sequence of interest, a transcriptional and translational termination region functional in plants. The termination region can be native with the DNA sequence of interest or can be derived from another source. Convenient termination regions are available from the Ti-plasmid of A. tumefaciens, such as the octopine synthase and nopaline synthase (nos) termination regions (Depicker et al., Mol. and Appl. Genet. 1:561-573 (1982); and Shaw et al. (1984), Nucleic Acids Research vol. 12, No. 20 pp. 7831-7846(nos)); see also Guerineau et al., Mol. Gen. Genet. 262:141-144 (1991); Proudfoot, Cell 64:671-674 (1991); Sanfacon et al., Genes Dev. 5:141-149 (1991); Mogen et al., Plant Cell 2:1261-1272 (1990); Munroe et al., Gene 91:151-158 (1990); Ballas et al., Nucleic Acids Res. 17:7891-7903 (1989); and Joshi et al., Nucleic Acid Res. 15:9627-9639 (1987).

An expression cassette may contain a 5′ leader sequence. Such leader sequences can act to enhance translation. Translation leaders are known in the art and include by way of example, picornavirus leaders, EMCV leader (Encephalomyocarditis 5′ noncoding region), Elroy-Stein et al., Proc. Nat. Acad. Sci. U.S.A. 86:6126-6130 (1989); potyvirus leaders, for example, TEV leader (Tobacco Etch Virus) Carrington and Freed, Journal of Virology, 64:1590-1597 (1990), MDMV leader (Maize Dwarf Mosaic Virus), Allison et al., Virology 154:9-20 (1986); human immunoglobulin heavy-chain binding protein (BiP), Macejak et al., Nature 353:90-94 (1991); untranslated leader from the coat protein mRNA of alfalfa mosaic virus (AMV RNA 4), Jobling et al., Nature 325:622-625 (1987); Tobacco mosaic virus leader (TMV), Gallie et al. (1989), Molecular Biology of RNA, pages 237-256; and maize chlorotic mottle virus leader (MCMV) Lommel et al., Virology 81:382-385 (1991). See also, Della-Cioppa et al., Plant Physiology 84:965-968 (1987).

The construct may also contain sequences that enhance translation and/or mRNA stability such as introns. An example of one such intron is the first intron of gene II of the histone H3.III variant of Arabidopsis thaliana. Chaubet et al., Journal of Molecular Biology, 225:569-574 (1992).

In those instances where it is desirable to have the expressed product of the heterologous nucleotide sequence directed to a particular organelle, particularly the plastid, amyloplast, or to the endoplasmic reticulum, or secreted at the cell's surface or extracellularly, the expression cassette may further comprise a coding sequence for a transit peptide. Such transit peptides are well known in the art and include, but are not limited to, the transit peptide for the acyl carrier protein, the small subunit of RUBISCO, plant EPSP synthase and Helianthus annuus (see, Lebrun et al., U.S. Pat. No. 5,510,417), Zea mays Brittle-1 chloroplast transit peptide (Nelson et al., Plant Physiol. 117(4):1235-1252 (1998); Sullivan et al., Plant Cell 3(12):1337-48; Sullivan et al., Planta (1995) 196(3):477-84; Sullivan et al., J. Biol. Chem. (1992), 267(26):18999-9004) and the like. In addition, chimeric chloroplast transit peptides are known in the art, such as the Optimized Transit Peptide (see, U.S. Pat. No. 5,510,471). Additional chloroplast transit peptides have been described previously in U.S. Pat. Nos. 5,717,084; 5,728,925. One skilled in the art will readily appreciate the many options available in expressing a product to a particular organelle. For example, the barley alpha amylase sequence is often used to direct expression to the endoplasmic reticulum. Rogers, J. Biol. Chem. 260:3731-3738 (1985).

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, stable integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno or Kozak sequences), modification of nucleic acid molecules to correspond to the codon usage of the host cell, and deletion of sequences that destabilize transcripts.

Reporter or marker genes for selection of transform ed cells or tissues or plant parts or plants may be included in the transformation vectors. Examples of selectable markers include those that confer resistance to anti-metabolites such as herbicides or antibiotics, for example, dihydrofolate reductase, which confers resistance to methotrexate (Reiss, Plant Physiol. (Life Sci. Adv.) 13:143-149, 1994; see also Herrera Estrella et al., Nature 303:209-213, 1983; Meijer et al., Plant Mol. Biol. 16:807-820, 1991); neomycin phosphotransferase, which confers resistance to the aminoglycosides neomycin, kanamycin and paromycin (Herrera-Estrella, EMBO J. 2:987-995, 1983 and Fraley et al., Proc. Natl. Acad. Sci. U.S.A. 80:4803 (1983)); hygromycin phosphotransferase, which confers resistance to hygromycin (Marsh, Gene 32:481-485, 1984; see also Waldron et al., Plant Mol. Biol. 5:103-108, 1985; Zhijian et al., Plant Science 108:219-227, 1995); trpB, which allows cells to utilize indole in place of tryptophan; hisD, which allows cells to utilize histinol in place of histidine (Hartman, Proc. Natl. Acad. Sci. U.S.A. 85:8047, 1988); mannose-6-phosphate isomerase which allows cells to utilize mannose (WO 94/20627); ornithine decarboxylase, which confers resistance to the ornithine decarboxylase inhibitor, 2-(difluoromethyl)-DL-ornithine (DFMO; McConlogue, 1987, In: Current Communications in Molecular Biology, Cold Spring Harbor Laboratory ed.); and deaminase from Aspergillus terreus, which confers resistance to Blasticidin S (Tamura, Biosci. Biotechnol. Biochem. 59:2336-2338, 1995).

Additional selectable markers include, for example, a mutant acetolactate synthase, which confers imidazolinone or sulfonylurea resistance (Lee et al., EMBO J. 7:1241-1248, 1988), a mutant psbA, which confers resistance to atrazine (Smeda et al., Plant Physiol. 103:911-917, 1993), or a mutant protoporphyrinogen oxidase (see U.S. Pat. No. 5,767,373), or other markers conferring resistance to an herbicide such as glufosinate. Examples of suitable selectable marker genes include, but are not limited to, genes encoding resistance to chloramphenicol (Herrera Estrella et al., EMBO J. 2:987-992, 1983); streptomycin (Jones et al., Mol. Gen. Genet. 210:86-91, 1987); spectinomycin (Bretagne-Sagnard et al., Transgenic Res. 5:131-137, 1996); bleomycin (Hille et al., Plant Mol. Biol. 7:171-176, 1990); sulfonamide (Guerineau et al., Plant Mol. Biol. 15:127-136, 1990); bromoxynil (Stalker et al., Science 242:419-423, 1988); glyphosate (Shaw et al., Science 233:478-481, 1986); phosphinothricin (DeBlock et al., EMBO J. 6:2513-2518, 1987), and the like.

One option for use of a selective gene is a glufosinate-resistance encoding DNA and in one embodiment can be the phosphinothricin acetyl transferase (pat), maize optimized pat gene or bar gene under the control of the Cassava Vein Mosaic Virus promoter. These genes confer resistance to bialaphos. See, Wohlleben et al. (1988), Gene 70: 25-37; Gordon-Kamm et al. (1990), Plant Cell 2:603; Uchimiya et al. (1993), BioTechnology 11:835; White et al. (1990), Nucl. Acids Res. 18:1062; Spencer et al. (1990), Theor. Appl. Genet. 79:625-631; and Anzai et al. (1989), Mol. Gen. Gen. 219:492. A version of the pat gene is the maize optimized pat gene, described in U.S. Pat. No. 6,096,947.

In addition, markers that facilitate identification of a plant cell containing the polynucleotide encoding the marker may be employed. Scorable or screenable markers are useful, where presence of the sequence produces a measurable product and can produce the product without destruction of the plant cell. Examples include a β-glucuronidase, or uidA gene (GUS), which encodes an enzyme for which various chromogenic substrates are known (for example, U.S. Pat. Nos. 5,268,463 and 5,599,670); chloramphenicol acetyl transferase (Jefferson et al., The EMBO Journal vol. 6 No. 13 pp. 3901-3907); and alkaline phosphatase. In a preferred embodiment, the marker used is beta-carotene or provitamin A (Ye et al., Science 287:303-305-(2000)). The gene has been used to enhance the nutrition of rice, but in this instance it is employed instead as a screenable marker, and the presence of the gene linked to a gene of interest is detected by the golden color provided. Unlike the situation where the gene is used for its nutritional contribution to the plant, a smaller amount of the protein suffices for marking purposes. Other screenable markers include the anthocyanin/flavonoid genes in general (see discussion at Taylor and Briggs, The Plant Cell (1990) 2:115-127) including, for example, a R-locus gene, which encodes a product that regulates the production of anthocyanin pigments (red color) in plant tissues (Dellaporta et al., in Chromosome Structure and Function, Kluwer Academic Publishers, Appels and Gustafson eds., pp. 263-282 (1988)); the genes which control biosynthesis of flavonoid pigments, such as the maize C1 gene (Kao et al., Plant Cell (1996) 8:1171-1179; Scheffler et al., Mol. Gen. Genet. (1994) 242:40-48) and maize C2 (Wienand et al., Mol. Gen. Genet. (1986) 203:202-207); the B gene (Chandler et al., Plant Cell (1989) 1:1175-1183), the p1 gene (Grotewold et al., Proc. Natl. Acad. Sci U.S.A. (1991) 88:4587-4591; Grotewold et al., Cell (1994) 76:543-553; Sidorenko et al., Plant Mol. Biol. (1999)39:11-19); the bronze locus genes (Ralston et al., Genetics (1988) 119:185-197; Nash et al., Plant Cell (1990) 2(11):1039-1049), among others.

Further examples of suitable markers include the cyan fluorescent protein (CYP) gene (Bolte et al. (2004), J. Cell Science 117: 943-54 and Kato et al. (2002), Plant Physiol. 129:913-42), the yellow fluorescent protein gene (PHIYFP™ from Evrogen; see Bolte et al. (2004), J. Cell Science 117:943-54); a lux gene, which encodes a luciferase, the presence of which may be detected using, for example, X-ray film, scintillation counting, fluorescent spectrophotometry, low-light video cameras, photon counting cameras or multiwell luminometry (Teeri et al. (1989), EMBO J. 8:343); a green fluorescent protein (GFP) gene (Sheen et al., Plant J. (1995) 8(5):777-84); and DsRed2 where plant cells transformed with the marker gene are red in color, and thus visually selectable (Dietrich et al. (2002), Biotechniques 2(2):286-293). Additional examples include a β-lactamase gene (Sutcliffe, Proc. Nat'l. Acad. Sci. U.S.A. (1978) 75:3737), which encodes an enzyme for which various chromogenic substrates are known (e.g., PADAC, a chromogenic cephalosporin); a xy1E gene (Zukowsky et al., Proc. Nat'l. Acad. Sci. U.S.A. (1983) 80:1101), which encodes a catechol dioxygenase that can convert chromogenic catechols; an α-amylase gene (Ikuta et al., Biotech. (1990) 8:241); and a tyrosinase gene (Katz et al., J. Gen. Microbiol. (1983) 129:2703), which encodes an enzyme capable of oxidizing tyrosine to DOPA and dopaquinone, which in turn condenses to form the easily detectable compound melanin. Clearly, many such markers are available and known to one skilled in the art.

### V. Assays for Detection of a Transgene or Expressed Product of a Transgene

Various assays can be employed to identify the location of the transformed transgene within the soybean stem sections. The following techniques are useful in a variety of situations, and in one embodiment, are useful in detecting the presence of the transgene and/or the polypeptide encoded by the transgene in a soybean plant stem. For example, plant material from the soybean stem section can be blotted onto a membrane and assayed for expression of the transgene. In an embodiment the plant material transferred to the membrane can be assayed via a Western blot to detect the protein expressed from the transgene. In another embodiment the plant material transferred to the membrane can be detected via an enzymatic assays. Further, an antibody which can detect the presence of the transgene can be generated and used to assay plant material from the soybean stem section that has been blotted onto a membrane and assayed for expression of the transgene. Additional techniques, such as in situ hybridization, enzyme staining, and immunostaining, also may be used to detect the presence or expression of the transgene from the soybean stem sections.

In the Western analysis, the soybean stem section is cut transversely and blotted directly to the membrane. The protein of interest which is expressed from the transgene that has been transformed into the soybean plants is transferred from the soybean stem section onto the membrane. The protein is contacted with a labeling substance, such as an antibody. See, e.g., Hood et al., “Commercial Production of Avidin from Transgenic Maize; Characterization of Transformants, Production, Processing, Extraction and Purification,” Molecular Breeding 3:291-306 (1997); Towbin et al. (1979), “Electrophoretic transfer of proteins from polyacrylamide gels to nitrocellulose sheets: procedure and some applications,” Proc. Natl. Acad. Sci. U.S.A. 76(9):4350-4354; Renart et al. “Transfer of proteins from gels to diazobenzyloxymethyl-paper and detection with antisera: a method for studying antibody specificity and antigen structure,” Proc. Natl. Acad. Sci. U.S.A. 76(7):3116-3120.

As used herein, the term “membrane” refers to a solid phase which is a porous or non-porous water insoluble material including, but not limited to, cellulose, polysaccharide such as SEPHADEX™, glass, polyacryloylmolpholide, silica, controlled pore glass (CPG), polystyrene, polystyrene/latex, polyethylene such as ultra-high molecular weight polyethylene (UPE), polyamide, polyvinylidine fluoride (PVDF), polytetrafluoroethylene (PTFE; TEFLON®), carboxyl modified TEFLON®, nylon, nitrocellulose, and metals and alloys such as gold, platinum and palladium. The membranes are charged and bind to organic materials such as proteins.

The membranes significantly improve Western blot transfer by making the process quantitative. By ensuring that the soybean stem sections are imprinted onto the member and are transferred to the adsorptive solid phase, it becomes possible to do quantitative studies to determine which tissues of the soybean stem section comprise the transformed transgene.

Another method for transferring the proteins is called electroblotting and uses an electric current to pull proteins from the stem section onto the membrane. Devices for electroblotting are commercially available. The proteins move from within the stem section onto the membrane while maintaining the organization they had within the gel. As a result of this “blotting” process, the proteins are exposed on a thin surface layer for detection. Both the secondary and absorptive membranes are selected for their non-specific protein binding properties (i.e., binds all proteins equally well). Protein binding is based upon hydrophobic interactions, as well as charged interactions between the membrane and protein.

The uniformity and overall effectiveness of transfer of protein from the gel to the membrane can be checked by staining the membrane with Coomassie or Ponceau S dyes, or other dyes. Coomassie is the more sensitive of the two, although the water solubility of Ponceau S makes it easier to subsequently destain and probe the membrane.

Since the membrane has been chosen for its ability to bind protein, and both antibodies and the target are proteins, steps must be taken to prevent interactions between the membrane and the antibody used for detection of the target protein. Blocking of non-specific binding is achieved by placing the membrane in a dilute solution of protein, typically Bovine serum albumin (BSA) or non-fat dry milk, with a minute percentage of detergent such as TWEEN® 20. The protein in the dilute solution attaches to the membrane in all places where the target proteins have not attached. Thus, when the antibody is added, there is no room on the membrane for it to attach other than on the binding sites of the specific target protein. This reduces “noise” in the final product of the Western blot, leading to clearer results, and eliminates false detection.

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

Proteins transferred to adsorptive membranes can be detected using a variety of conventional techniques. A preferred detection technique uses antibodies specific for a particular polypeptide. The primary antibody can be detected using any one of several techniques. A variety of methods to detect specific antibody-antigen interactions are known in the art and can be used in the method, including, but not limited to, standard immunohistological methods, immunoprecipitation, an enzyme immunoassay, and a radioimmunoassay. In general, the polypeptide-specific antibody will be detectably labeled, either directly or indirectly. Direct labels include radioisotopes; enzymes whose products are detectable (e.g., luciferase, β-galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, and aequorin (green fluorescent protein).

The adsorptive membranes containing transferred proteins may then be washed with suitable buffers, followed by contacting with a detectably-labeled polypeptide-specific antibody. Detection methods are known in the art and will be chosen as appropriate to the signal emitted by the detectable label. Detection is generally accomplished in comparison to suitable controls, and to appropriate standards.

In an embodiment the Western blotting can be utilized for tissue printing of the soybean stem sections to identify germline and non-germline soybean transformants. Germline soybean transformants can be identified by the “dot pattern” which is produced on the membrane following detection with an antibody of other label. The dot pattern appears on the membrane as a solid filled circle. Comparatively, the non-germline soybean transformants can be identified by the “ring pattern” which is produced on the membrane following detection with an antibody or other label. The ring pattern appears on the membrane as a circular line. The center of the ring pattern is devoid of signal from an antibody or other label.

Accordingly, the “dot pattern” is indicative of soybean plant stems which are transformed with a transgene in the core tissue (L2 and L3 layer) and which will result in germline soybean transformants. Comparatively, the “ring pattern” is indicative of soybean plant stems which are transformed with a transgene in the mantle tissue (L1 layer) and which will result in non-germline soybean transformants.

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1

### DNA Construct

A single binary vector labeled as pDAB9381 (FIG. 1) was constructed using art recognized procedures. pDAB9381 contains two Plant Transcription Units (PTUs). The first PTU (SEQ ID NO:1) consists of the Arabidopsis thaliana ubiquitin-10 promoter (AtUbi10 promoter; J. Callis, et al. (1990), Ubiquitin extension proteins of Arabidopsis thaliana. Structure, localization and expression of their promoters in transgenic tobacco. J. Biol. Chem. 265:12486-12493) which drives the yellow fluorescence protein coding sequence (PhiYFP; Shagin, et al. (2004), GFP-like proteins as ubiquitous metazoan superfamily: evolution of functional features and structural complexity, Molecular Biology and Evolution 21(5):841-850) that contains an intron isolated from the Solanum tuberosum, light specific tissue inducible LS-1 gene (ST-LS1 intron; Genbank Acc No. X04753), and is terminated by the Agrobacterium tumefaciens open reading frame-23 3′ untranslated region (AtuORF23 3′UTR; Gelvin SG (1987) TR-based sub-Ti plasmids, EP Patent 222493). The second PTU (SEQ ID NO:2) was cloned within the isopentenyltransferase coding sequence (ipt CDS; Genbank Acc No. X00639.1), consisting of the Cassava Vein Mosaic Virus promoter (CsVMV promoter; B. Verdaguer, et al. (1996), Isolation and expression in transgenic tobacco and rice plants of the cassava vein mosaic (CVMV) promoter, Plant Mol. Biol. 31:1129-1139) which is used to drive the phosphinothricin acetyl transferase coding sequence (PAT; W. Wohlleben et al. (1988), Nucleotide sequence of the phosphinothricin N-acetyl-transferase gene from Streptomyces viridochromogenes Tu494 and its expression in Nicotiana tobacum, Gene 70:25-38), terminated by the A. tumefaciens open reading frame-1 3′ untranslated region (AtuORF1 3′UTR; M. L. Huang et al. (1990), A chromosomal Agrobacterium gene required for effective plant signal transduction, J. Bacteriol. 172:1814-1822). The resulting binary vector contained a visual reporter gene and an antibiotic selectable marker gene and was subsequently used for the transformation of soybean.

### Example 2

### Transformation of Soybean

Two methods for the Agrobacterium-mediated transformation of soybean were employed. These methods include the cotyledonary node transformation method and split-seed transformation method. Both protocols are described in more detail below.

Transformation Method 1: Cotyledonary node transformation of soybean mediated by Agrobacterium tumefaciens.

Agrobacterium tumefaciens strain EHA105 (E. Hood, G. Helmer, R. Fraley, M. Chilton (1986), J. Bacteriol. 168:1291-1301) was electroporated with the binary vector pDAB9381. Isolated colonies were identified which grew up on YEP media containing the antibiotic spectinomycin. Single colonies were isolated and the presence of the pDAB9381 binary vector was confirmed via restriction enzyme digestion. Agrobacterium-mediated transformation of soybean (Glycine max c.v., Maverick) was performed using the pDAB9381 binary vector via a modified procedure of P. Zeng et al. (2004), Plant Cell Rep. 22(7):478-482. The protocol was modified to include the herbicide glufosinate as a selective agent. In addition, another modification included the germination of sterilized soybean seeds on B5 basal medium (Gamborg et al. (1968), Exp. Cell Res. Apr 50(1):151-8) solidified with 3 g/L Phytagel (Sigma-Aldrich, St. Louis, Mo.). The final modification to the protocol deployed the use of cotyledonary node explants that were prepared from 5-6 days old seedlings and infected with Agrobacterium as described by Zhang et al. (1999), The use of glufosinate as a selective agent in Agrobacterium-mediated transformation of soybean, Plant Cell, Tissue, and Organ Culture 56:37-46.

As described in Zeng et al. (2004), co-cultivation was carried out for 5 days on the co-cultivation medium. Shoot initiation, shoot elongation, and rooting media were supplemented with 50 mg/L cefotaxime, 50 mg/L timentin, 50 mg/L vancomycin, and solidified with 3 g/L Phytagel. Selected shoots were then transferred to the rooting medium.

Transformation Method 2: Split-Seed Transformation of Soybean Mediated by Agrobacterium tumefaciens

The split-seed transformation method described below is described more fully in U.S. Provisional Patent Application No. 61/739,349 filed Dec. 19, 2012, the entire disclosure of which is incorporated here by reference. In brief, Agrobacterium tumefaciens strain EHA105 (Hood et al., 1986) was electroporated with the binary vector pDAB9381. Isolated colonies were identified which grew up on YEP media containing the antibiotic spectinomycin. Single colonies were isolated and the presence of the pDAB9381 binary vector was confirmed via restriction enzyme digestion.

Agrobacterium-mediated transformation of soybean (Glycine max c.v. Maverick) was performed using the pDAB9381 binary vector via the modified procedure of M. Paz, et al. (2005), Plant Cell Rep. 25:206-213. Briefly, mature soybean seeds were sterilized overnight with chlorine gas, and imbibed with sterile H₂O sixteen hours in the dark using a black box at 24° C. before Agrobacterium-mediated plant transformation. Seeds were cut in half by a longitudinal cut along the hilum to separate the seed and remove the seed coat. The embryonic axis was partially excised and any axial shoots/buds were removed from the cotyledonary node.

The sterilized seeds were inoculated for 30 minutes in an Agrobacterium tumefaciens strain EHA105 harboring the pDAB9381 binary vector. Next, the explants were allowed to co-cultivate with the Agrobacterium tumefaciens strain for 5 days on co-cultivation medium covered with a filter paper. After 5 days of co-cultivation, the explants were washed in liquid shoot induction (SI) medium containing 100 mg/L Timentin, 200 mg/L Cefotaxime, and 50 mg/L Vancomycin. The explants were then cultured on semi-solid Shoot Induction 1 (SI-1) medium (produced by adding a gelling agent to SI medium), wherein the flat side of the soybean seed was place up with nodal end of the soybean cotyledon imbedded into the medium.

After 2 weeks of culture on the SI-1 medium at 24° C. with a 18 hour photoperiod (illumination of 80-90 μmole m⁻² sec⁻¹), the explants were transferred to the Shoot Induction 2 (SI-2) medium which was formulated from the SI-1 medium by supplementing it with 6 mg/L glufosinate. After 2 weeks on SI-2 medium, the cotyledons were removed from the explants and the explants with developing shoots were excised by making a cut at the base and transferred to the Shoot Elongation (SE) medium. The cultures were transferred to fresh SE medium every 2 weeks until all the shoots were regenerated. The number of transfers varied from 6-8 transfers, for a duration of 12-16 weeks.

The above-described soybean transformation protocols produced transgenic soybean events that were either germline or non-germline transformants. Since in most angiosperms, the germ cells are formed from L2/L3 layer cells, germline soybean transformants result when the transgene is inserted into the genomic DNA of the L2/L3 layer cells of the shoot apical meristem. Germline transformants are capable of passing a transgene on to progeny plants (i.e., T₁, T₂, etc.) and are suitable for conventional commercial cultivation. Non-germline soybean transformants result when the transgene is inserted into the genomic DNA of only the L1 layer cells of the shoot apical meristem, which can only divide anticlinally, forming the epidermal cell layer of the plant. Non-germline transformants do not pass the transgene on to the progeny plants (i.e., T₁, T₂, etc.) and so are undesirable. The next set of Examples describes a novel screening method which was used to identify and advance the germline transformants, and to detect and eliminate the non-germline transformants.

### Example 3

### Tissue Printing

Soybean shoots which reached a length of 2.5 cm were selected from the shoot elongation (SE) media. A clean horizontal cut (transverse stem section) was made to excise stem sections from the base of the shoots. Soybean stem sections of approximately 0.1-0.5 mm thickness were excised and collected from the shoots. One of the isolated stem sections was pressed onto a piece of nitrocellulose membrane (Bio-Rad, Hercules, Calif.), forming a “tissue print” of the stem cross section. The stem section was pressed against the nitrocellulose membrane for 15 seconds using gentle pressure from a gloved finger. This process was repeated for each stem section that was isolated from an individual soybean shoot. The soybean shoot and the location of the corresponding stem sections on the nitrocellulose membrane were recorded. In addition, 5 μL of a 1 ng/μL stock solution of purified PhiYFP protein was dotted onto the nitrocellulose membrane in the lower right corner of the nitrocellulose membrane as a positive control. Next, the nitrocellulose membrane was allowed to air dry at room temperature for 10 minutes. Finally, the nitrocellulose membrane was soaked in deionized water for 5 minutes, and analyzed via a Western blot procedure.

Western blotting was completed by blocking the nitrocellulose membrane for 30 minutes in Blocking Solution (2% milk solids suspended in phosphate buffered saline containing 0.05% TWEEN® 20 [PBST]). Next, the nitrocellulose membrane was washed two times for 5 minutes in PBST. The nitrocellulose membrane was immersed into a solution of 2% milk solids suspended in PBST and a primary antibody (polyclonal rabbit anti-PhiYFP at 1 μg/mL (Evrogen, Moscow, Russia) was added and the mixture was incubated with gentle shaking at room temperature for 60 minutes. Next, the nitrocellulose membrane was washed four times for 5 minutes in PBST. The nitrocellulose membrane was immersed into a solution of 2% milk solids suspended in PBST and a secondary antibody (horse radish peroxidase conjugated goat anti-rabbit IgG in PBST (Sigma, St. Louis, Mo.) was added and the mixture was incubated with gentle shaking for 30 minutes. Next, the nitrocellulose membrane was washed four times for 5 minutes in PBST. Finally, the nitrocellulose membrane was washed three times for 2 minutes in PBS.

Excess moisture was removed from the nitrocellulose membrane by lightly patting the corner of the membrane on a paper towel. Antibody binding was detected using ECL Plus™ chemiluminescent detection reagents (GE Healthcare, Waukesha, Wis.) following the manufacturer's directions. The chemiluminescence which emanated from the antibodies bound to the nitrocellulose membrane was visualized using a G:Box™ gel documentation and analysis system (SynGene, Frederick, Md.). An exposure time of 10 minutes and “no binning” were the parameters used for the G:Box™ documentation and analysis of the nitrocellulose membrane. Selection of “no binning” of the image was necessary to maintain resolution and correctly identify the signal pattern from the stem section tissue prints.

Soybean shoots which contained a functionally expressing copy of the PhiYFP transgene produced tissue prints with two distinct image patterns. These image patterns were classified as either “rings” or “dots.” FIG. 2 shows a photographic image of the two observed patterns. The ring pattern is characterized as an open circle, comprised of a circular outline of chemiluminescent signal which is devoid of chemiluminescent signal within the center of the stem section tissue print (FIG. 2, Panel a). The dot pattern is characterized as a filled circle, comprised of a chemiluminescent signal throughout the entire stem section tissue print (FIG. 2, Panel b). An image of the membrane using epi-white light illumination was taken to compare positions of the soybean stem section prints with the patterns from the chemiluminescence image. By correlating the epi-white light illumination image with the chemiluminescence image, the specific soybean shoot that was used to generate the stem section print could be identified and classified as either a ring or dot image pattern. (FIG. 2, Panels c and d.)

Transgenic soybean events that were germline transformants produced tissue prints that possessed a dot pattern. Comparably, transgenic soybean events that were non-germline transformants produced tissue prints that possessed a ring pattern. The dot pattern resulted from the insertion of the transgene into the genomic DNA of the cells of the L2 and L3 layers (which divide to form the cortex, vasculature, and pith). The ring pattern resulted from the insertion of the transgene into the genomic DNA of the L1 layer cells (which divide to form the epidermal cell layer). This detection method can be used to differentiate germline soybean transformants from non-germline soybean transformants. A method which allows for the identification of germline transformants is desirable as transgenic events which are capable of passing a transgene onto progeny plants are identified earlier within the transformation process, thereby reducing the total number of transgenic events which must be advanced through the transformation process. The early identification of germline transformants (in which imprinted soybean stem sections produce a “dot” pattern) results in a more efficient soybean transformation pipeline.

### Example 4

### Confocal Microscopy

Confocal microscopy was used to image the second excised soybean stem section. These stem sections were mounted on glass coverslips and viewed with a Leica SP5 confocal microscope (Wetzlar, Germany). The stem sections were illuminated with the 514 nm line of an argon-ion laser. Emission data of the PhiYFP protein, which was expressed in the soybean stem section samples, was collected from between 530-540 nm.

In general, the fluorescence patterns of PhiYFP protein expression from the soybean stem samples were categorized into one of three classes: a) no fluorescence; b) fluorescence of tissue derived from the L1 layer of the meristem (the epidermal layer); and c) fluorescence tissue derived from the L2/L3 layers of the meristem (the cortex, vascular tissues, and pith). Examples of three classes of fluorescence patterns from the soybean stem samples are shown in FIG. 3. Transgenic soybean events that were germline (L2/L3 layers) transformants produced fluorescence in the cortex, vascular tissues, and pith of the stem. The fluorescence resulting from the insertion of the transgene into the genomic DNA of the cells of the L2/L3 layer can be identified by observing the expression of the PhiYFP protein within the cortex, vascular tissue, and pith of the stem. Comparably, transgenic soybean events that were non-germline (L1 layer) transformants produced fluorescence in only the epidermal cells. The fluorescence resulting from the insertion of the transgene into the genomic DNA of the cells of the L1 layer can be identified by observing the expression of the PhiYFP protein within the epidermal cells of the stem. This detection method can be used to identify germline soybean transformants from non-germline soybean transformants. The identification of germline transformants is desirable as transgenic events which are capable of passing a transgene onto progeny plants are identified earlier within the transformation process, thereby reducing the total number of transgenic events which must be advanced through the transformation process. The early identification of germline transformants results in a more efficient soybean transformation pipeline.

### Example 5

### Correlation of Confocal Microscopy Observations and Tissue Printing Results

The results from the tissue printing of the stem sections which produced the dot image patterns were found to correlate with the confocal microscopy observations of the stem sections which expressed PhiYFP within the cortex, vascular tissue, and pith of the stem. 85% of the soybean shoot samples (6 out of 7) showing cortex expression in confocal microscopy observations also exhibited the dot image pattern which indicated that the transgene had inserted into the genomic DNA of the L2/L3 layers. As such, these soybean shoot samples could be labeled as germline transformants, and advanced through the soybean transformation process. There was less of a correlation (29%) between the stem sections which produced the ring image patterns for tissue printing as compared to stem sections which expressed PhiYFP within the epidermal cell layer (L1 layer transformants), as observed by confocal microscopy. This percentage is lower as the tissue printing method did not produce ring image patterns for a number of soybean shoots in which the confocal microscopy results indicated that the PhiYFP protein was expressing only within the epidermal cell layer (L1 layer transformants). However, the tissue printing results only exhibited one false negative result, in which a ring pattern observed in the tissue printing procedure was not confirmed as epidermal expression by confocal microscopy. The correlation between the stem sections which produced the ring image patterns for tissue printing, as compared to stem sections which expressed PhiYFP within the epidermal cell layers as observed by confocal microscopy can be improved by modifying the tissue printing method to improve the sensitivity of PhiYFP detection within the epidermal cell layer. Importantly, however, there was no instance in this dataset of a failure to observe a pattern in the tissue printing analysis that was later found by confocal microscopy to correlate to cortex expression. The results of the tissue printing analyses and the confocal microscopy observations of the stem sections which expressed PhiYFP are given in Table 1.

TABLE 1
Summary of results from confocal microscopy and tissue printing
analyses. Confocal microscopy results labeled as “interior
tissue expression” were samples which produced fluorescence
in the cortex, vascular tissues, and pith cell layers of the stem.
Samples marked “Interior tissue expression” may also
have fluorescence in the epidermis, in addition to the interior tissues.
Confocal microscopy results labeled as “Epidermal expression”
were samples which produced fluorescence in the epidermal
cell layer only.

| Sample | Confocal Results | Tissue Printing |
|---|---|---|
| 1 | Epidermal only expression | No pattern |
| 2 | No fluorescence | No pattern |
| 3 | Epidermal only expression | No pattern |
| 4 | Interior tissue expression | Ring pattern |
| 5 | Interior tissue expression | Dot pattern |
| 6 | Epidermal only expression | No pattern |
| 7 | No fluorescence | No pattern |
| 8 | Epidermal only expression | Ring pattern |
| 9 | Interior tissue expression | Ring pattern |
| 10 | Epidermal only expression | Dot pattern |
| 11 | Epidermal only expression | No pattern |
| 12 | Epidermal only expression | No pattern |
| 13 | No fluorescence | No pattern |
| 14 | Interior tissue expression | Dot pattern |
| 15 | Epidermal only expression | No pattern |
| 16 | Epidermal only expression | Ring pattern |
| 17 | Interior tissue expression | Dot pattern |
| 18 | Epidermal only expression | Ring pattern |
| 19 | Epidermal only expression | No pattern |
| 20 | Epidemial only expression | No pattern |
| 21 | Interior tissue expression | Dot pattern |
| 22 | Interior tissue expression | Dot pattern |
| 23 | Interior tissue expression | Dot pattern |
| 24 | Epidermal only expression | No pattern |

### Example 6

### Generation of T₀ Plants and T₁ Seed Production

Prior to transferring elongated shoots (3-5 cm) to rooting medium, the excised end of the shoot internodes were dipped in 1 mg/L indole 3-butyric acid for 1-3 min to promote rooting (Khan et al. (1994), Agrobacterium-Mediated Transformation of Subterranean Clover (Trifolium subterraneum L.), Plant Physiol., May 105(1):81-88). The soybean shoots that generated roots in 25×100 mm glass culture tubes containing rooting medium were transferred to soil mix in open Magenta boxes and placed within a Conviron™ for acclimatization of plantlets. Glufosinate, the active ingredient of Liberty herbicide (Bayer Crop Science), was used for selection during shoot initiation and elongation. The rooted plantlets were acclimated in open Magenta boxes for several weeks before they were molecularly screened and transferred to the greenhouse for further acclimation and establishment. The resulting transgenic soybean plants were grown in the greenhouse and allowed to self-fertilize for T₁ seed production.

### Example 7

### Molecular Characterization of Transformants Via Hydrolysis and qPCR

The presence of the transgenes within the genome of soybean transformants which were transformed with pDAB9381 was continued. The soybean transformants were initially screened via a hydrolysis probe assay, analogous to TAQMAN™, to confirm the presence of the pat and yfp transgenes. Soybean transformants were screened to confirm the presence and to estimate the copy number transgenes within the plant chromosome.

Hydrolysis Probe Assay

Copy number was determined in the T₀ and T₁ Glycine max plants using the hydrolysis probe assay described below. Plants with varying numbers of transgenes were identified.

Tissue samples were collected using a hole punch and placed in 96-well plates. A tissue sample from a fully expanded top leaf and fully expanded bottom leaf of each plant were chosen to analyze separately to address transformation chimerism in T₀ Glycine max plants. The genomic DNA was isolated in high-throughput format using the Agilent BioCel (Agilent, Santa Clara, Calif.) and the Qiagen MagAttract DNA Isolation Kit™ (Qiagen, Germantown, Md.). Hydrolysis probe assays were performed to determine transgene copy number of PATv6 and presence/absence of YFP, in relation to an internal reference gene, GMS116 by real-time PCR using the LIGHTCYCLER®480 system (Roche Applied Science, Indianapolis, Ind.).

For amplification of PATv6, LIGHTCYCLER®480 Probes Master mix (Roche Applied Science, Indianapolis, Ind.) was prepared at a 1× final concentration in a 10 μL volume multiplex reaction containing 0.4 μM of each primer for PATv6 and 0.4 μM of each primer for GMS116 and 0.2 μM of each probe (Table 2). A two-step amplification reaction was performed with an extension at 60° C. for 60 seconds with fluorescence acquisition. All samples were run and the Cycle threshold (Ct) values were used for analysis of each sample. Analysis of real time PCR data was performed using LIGHTCYCLER® software release 1.5 using the relative quant module and is based on the ΔΔCt method. For this, a sample of genomic DNA from known 4, 2, 1 and 0.5 copy number checks were included in each run. The copy number results of the hydrolysis probe screen were determined for the T₀ and T₁ transgenic Glycine max plants.

TABLE 2
Primer and probe Information for hydrolysis probe assay of PATv6
and internal reference gene (GMS116).

| Primer Name | SEQ ID NO: | Sequence |
|---|---|---|
| PATv6-F | SEQ ID NO: 3 | 5′-ACAAGAGTGGATTGATGATCTAGAGA-3′ |
| PATv6-R | SEQ ID NO: 4 | 5′-CTTTGATGCCTATGTGACACGTAAAC-3′ |
| PATv6-FAM probe | SEQ ID NO: 5 | 5′/56-FAM/CAAGGCGTAAGCAATACCAGC/3BHQ_1/3′ |
| GMS116-F | SEQ ID NO: 6 | 5′-GTAATATGGGCTCAGAGGAATGGT-3′ |
| GMS116-R | SEQ ID NO: 7 | 5′-ATGGAGAAGAACATTGGAATTGC-3′ |
| GMS116-FAM probe | SEQ ID NO: 8 | 5′HEX/CCATGGCCCGGTACCATCTGGTC/3BHQ_1/3′ |

For amplification of YFP, LIGHTCYCLE®480 Probes Master mix (Roche Applied Science, Indianapolis, Ind.) was prepared at a 1× final concentration in a 10 μL volume multiplex reaction containing 0.4 μM of each primer for YFP and 0.4 μM of each primer for GMS116 and 0.2 μM of each probe (Table 3). A two-step amplification reaction was performed with an extension at 60° C. for 60 seconds with fluorescence acquisition. All samples were run and the Cycle threshold (Ct) values were used for analysis of each sample. Analysis of real time PCR data was performed using LIGHTCYCLER® software release 1.5 using the relative quant module and is based on the ΔΔCt method. For this, a sample of known positive sample, plasmid positive control, wild type genomic DNA negative control, and water (no amplification) control were used in each run. The presence/absence results of the hydrolysis probe screen were determined for the T₀ and T₁ transgenic Glycine max plants.

TABLE 3
Primer and probe Information for hydrolysis probe assay of YFP and
internal reference gene (GMS116).

| Primer Name | SEQ ID NO: | Sequence |
|---|---|---|
| YFP-F | SEQ ID NO: 9 | 5′-CGTGTTGGGAAAGAACTTGGA-3′ |
| YFP-R | SEQ ID NO: 10 | 5′-CCGTGGTTGGCTTGGTCT-3′ |
| YFP-FAM probe | SEQ ID NO: 11 | 5′/6-FAM/CACTCCCCACTGCCT/MGBNFQ/3′ |
| GMS116-F | SEQ ID NO: 12 | 5′-GTAATATGGGCTCAGAGGAATGGT-3′ |
| GMS116-R | SEQ ID NO: 13 | 5′-ATGGAGAAGAACATTGGAATTGC-3′ |
| GMS116-FAM probe | SEQ ID NO: 14 | 5′HEX/CCATGGCCCGGTACCATCTGGTC/3BHQ_1/3′ |

### Example 8

### T₁ Molecular Analysis Correlates with T₀ Confocal Microscopy and Tissue Imprinting Data

The confocal and tissue printing results of the T₀ shoot sections can be used to identify germline transformants. The confocal and tissue printing results of the T₀ shoot sections correlated with the molecular data which was obtained from the T₁ progeny soybean plants. A total of 19 transgenic events consisting of the T₀ and T₁ progeny plants are summarized in Table 4. There is a correlation between the T₀ tissue printing data and T₁ molecular data. Of the soybean events which were screened by confocal microscopy and tissue printing methods, 80% (4 out of 5) of the soybean events demonstrated that tissue printing (events possessed a dot image pattern) and confocal microscopy (fluorescence of the protein was observed in the cortical tissues) can be used early in the plant transformation process to identify and predict which events were germline transformants prior to transferring the soybean shoots to rooting medium. Likewise, the confocal microscopy and tissue printing methods can be used to identify non-germline transformants. 92% (11 out of 12) of the soybean events demonstrated that tissue printing (events possessed a ring or no image pattern) and confocal microscopy (fluorescence of the protein was observed in the epidermal tissues) can be used early in the plant transformation process to identify and predict which events were non-germline transformants. In other words, tissue printing analysis returned no false positive results, and only returned one false negative result in this dataset. The early identification of the non-germline transformants allows for these events to be removed from the soybean transformation process. By removing these non-germline transformants, more resources can be devoted to germline soybean transformants which are capable of passing the inserted transgene to progeny plants.

Table 4: Analysis of soybean events transformed with pDAB9381. Tissue printing and confocal microscopy data of T₀ plants as compared to the molecular analysis of T₁ progeny plants.

TABLE 4
Analysis of soybean events transformed with pDAB9381. Tissue
printing and confocal microscopy data of T₀ plants as compared
to the molecular analysis of T₁ progeny plants.

| | T₀ | | T₁ |
|---|---|---|---|
| | Confocal | | PAT/PhiYFP |
| Event # | Microscopy | Tissue Printing | Presence |
| 5477[205]-2601.001 | epidermis | No | No |
| 5477[205]-2643.001 | epidermis | No | No |
| 5477[205]-2644.001 | epidermis | No | No |
| 5477[206]-2604.001 | cortex | Dot | Yes |
| 5477[206]-2605.001 | cortex | Dot | Yes |
| 5477[206]-2646.001 | epidermis | Ring | No |
| 5477[208]-2607.001 | epidermis | No | No |
| 5477[208]-2650.001 | cortex | Dot | No |
| 5477[208]-2651.001 | epidermis | Ring | Yes |
| 5477[208]-2652.001 | epidermis | No | No |
| 5477[208]-2653.001 | cortex | Dot | No |
| 5477[209]-2655.001 | epidermis | No | No |
| 5477[209]-2656.001 | none | None | No |
| 5477[209]-2657.001 | epidermis | No | No |
| 5477[209]-2658.001 | cortex | Ring | Yes |
| 5477[209]-2659.001 | epidermis | No | No |
| 5477[209]-2660.001 | none | No | No |
| 5477[209]-2661.001 | epidermis | Ring | No |
| 5477[209]-2662.001 | epidermis | Ring | No |

While aspects of this invention have been described in certain embodiments, they can be further modified within the spirit and scope of this disclosure. This application is therefore intended to cover any variations, uses, or adaptations of embodiments of the invention using its general principles. Further, this application is intended to cover such departures from the present disclosure as come within known or customary practice in the art to which these embodiments pertain and which fall within the limits of the appended claims.

What is claimed is:

1. A method for identifying a soybean germline transformant comprising the steps of:
(a) transforming a soybean plant tissue with a transgene;
(b) regenerating a shoot from the transformed soybean plant tissue comprising the transgene;
(c) isolating the regenerated shoot from the transformed soybean plant tissue comprising the transgene;
(d) bringing the isolated regenerated shoot into contact with a membrane, wherein a plant material comprising the transformed soybean plant tissue comprising the transgene is transferred from the isolated regenerated shoot and fixed to the membrane;
(e) assaying the transferred plant material to determine a location of the transgene within the transferred plant material;
(f) determining a transgene location within the isolated regenerated shoot, wherein the transgene location is selected from the group consisting of a L2/L3 tissue layer and a L1 tissue layer; and
(g) identifying an isolated regenerated shoot exhibiting a L2/L3 tissue layer transgene location as a soybean germline transformant.

2. A method of regenerating a mature transgenic soybean plant from the soybean germline transformant of claim 1, comprising:
(a) selecting the isolated regenerated shoot comprising the soybean germline transformant; and
(b) culturing the isolated regenerated shoot comprising the soybean germline transformant into a mature transgenic soybean plant.

3. The method of claim 1, wherein the transforming employs a transformation method elected from the group consisting of Agrobacterium transformation, biolistics, calcium phosphate transformation, polybrene transformation, protoplast fusion transformation, electroporation transformation, ultrasonic transformation, liposome transformation, microinjection transformation, naked DNA transformation, plasmid vector transformation, viral vector transformation, silicon carbide mediated transformation, aerosol beaming transformation, or PEG transformation.

4. The method of claim 1, wherein the soybean plant tissue is selected from the group consisting of a meristematic soybean plant tissue, a dermal soybean plant tissue, a ground soybean plant tissue, and a vascular soybean plant tissue.

5. The method of claim 4, wherein the meristematic soybean plant tissue comprises apical meristem, primary meristem, or lateral meristem.

6. The method of claim 4, wherein the dermal soybean plant tissue comprises epidermis.

7. The method of claim 4, wherein the dermal soybean plant tissue comprises periderm.

8. The method of claim 4, wherein the vascular soybean plant tissue is selected from the group consisting of xylem or phloem.

9. The method of claim 1, wherein the transgene is contained within at least one gene expression cassette.

10. The method of claim 9, wherein the gene expression cassette comprises a selectable marker gene.

11. The method of claim 9, wherein the gene expression cassette comprises a trait gene.

12. The method of claim 9, wherein the gene expression cassette comprises an RNAi gene.

13. The method of claim 1, wherein the isolating comprises making a horizontal cut transverse to the shoot.

14. The method of claim 1, wherein the membrane comprises a nitrocellulose membrane, a nylon membrane, a polytetrafluoroethylene membrane, or a polyvinylidene fluoride membrane.

15. The method of claim 1, wherein the assaying comprises a protein detection assay.

16. The method of claim 15, wherein the protein detection assay comprises an antibody that binds specifically to a protein expressed from the transgene.

17. The method of claim 1, wherein the determining comprises recognizing a dot pattern within the transferred plant material fixed to the membrane.

18. The method of claim 1, wherein the determining comprises recognizing a ring pattern within the transferred plant material fixed to the membrane.


SEQUENCE LISTING (14 sequences)

SEQ ID NO: 1 - DNA, 2783 bp, Artificial Sequence
  YFP plant transcription unit
gtcgacctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacagagatctccatgtcatctggagcacttctctttcatgggaagattccttacgttgtggagatggaagggaatgttgatggccacacctttagcatacgtgggaaaggctacggagatgcctcagtgggaaaggtatgtttctgcttctacctttgatatatatataataattatcactaattagtagtaatatagtatttcaagtatttttttcaaaataaaagaatgtagtatatagctattgcttttctgtagtttataagtgtgtatattttaatttataacttttctaatatatgaccaaaacatggtgatgtgcaggttgatgcacaattcatctgtactaccggagatgttcctgtgccttggagcacacttgtcaccactctcacctatggagcacagtgctttgccaagtatggtccagagttgaaggacttctacaagtcctgtatgccagatggctatgtgcaagagcgcacaatcacctttgaaggagatggcaacttcaagactagggctgaagtcacctttgagaatgggtctgtctacaatagggtcaaactcaatggtcaaggcttcaagaaagatggtcacgtgttgggaaagaacttggagttcaacttcactccccactgcctctacatctggggagaccaagccaaccacggtctcaagtcagccttcaagatatgtcatgagattactggcagcaaaggcgacttcatagtggctgaccacacccagatgaacactcccattggtggaggtccagttcatgttccagagtatcatcatatgtcttaccatgtgaaactttccaaagatgtgacagaccacagagacaacatgagcttgaaagaaactgtcagagctgttgactgtcgcaagacctacctttgagtagttagcttaatcacctagagctcggtcaccagcataatttttattaatgtactaaattactgttttgttaaatgcaattttgctttctcgggattttaatatcaaaatctatttagaaatacacaatattttgttgcaggcttgctggagaatcgatctgctatcataaaaattacaaaaaaattttatttgcctcaattattttaggattggtattaaggacgcttaaattatttgtcgggtcactacgcatcattgtgattgagaagatcagcgatacgaaatattcgtagtactatcgataatttatttgaaaattcataagaaaagcaaacgttacatgaattgatgaaacaatacaaagacagataaagccacgcacatttaggatattggccgagattactgaatattgagtaagatcacggaatttctgacaggagcatgtcttcaattcagcccaaatggcagttgaaatactcaaaccgccccatatgcaggagcggatcattcattgtttgtttggttgcctttgccaacatgggagtccaaggtt

SEQ ID NO: 2 - DNA, 1828 bp, Artificial Sequence
  PAT plant transcription unit
ccagaaggtaattatccaagatgtagcatcaagaatccaatgtttacgggaaaaactatggaagtattatgtaagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtaggtaccagatctggatcccaaaccatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatctacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccaaatctgagtagttagcttaatcacctagagctcgatcggcggcaatagcttcttagcgccatcccgggttgatcctatctgtgttgaaatagttgcggtgggcaaggctctctttcagaaagacaggcggccaaaggaacccaaggtgaggtgggctatggctctcagttccttgtggaagcgcttggtctaaggtgcagaggtgttagcgggatgaagcaaaagtgtccgattgtaacaagatatgttgatcctacgtaaggatattaaagtatgtattcatcactaatataatcagtgtattccaatatgtactacgatttccaatgtctttattgtcgccgtatgtaatcggcgtcacaaaataatccccggtgactttcttttaatccaggatgaaataatatgttattataatttttgcgatttggtccgttataggaattgaagtgtgcttgaggtcggtcgccaccactcccatttcataattttacatgtatttgaaaaataaaaatttatggtattcaatttaaacacgtatacttgtaaagaatgatatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatggtgtaatacatagg

SEQ ID NO: 3 - DNA, 26 bp, Artificial Sequence
  PATv6 - F primer sequence
acaagagtggattgatgatctagaga

SEQ ID NO: 4 - DNA, 26 bp, Artificial Sequence
  PATv6 - R primer sequence
ctttgatgcctatgtgacacgtaaac

SEQ ID NO: 5 - DNA, 21 bp, Artificial Sequence
  PATv6 - FAM probe sequence
caaggcgtaagcaataccagc

SEQ ID NO: 6 - DNA, 24 bp, Artificial Sequence
  GMS116 - F primer sequence
gtaatatgggctcagaggaatggt

SEQ ID NO: 7 - DNA, 23 bp, Artificial Sequence
  GMS116 - R primer sequence
atggagaagaacattggaattgc

SEQ ID NO: 8 - DNA, 23 bp, Artificial Sequence
  GMS116 - FAM probe sequence
ccatggcccggtaccatctggtc

SEQ ID NO: 9 - DNA, 21 bp, Artificial Sequence
  YFP - F primer sequence
cgtgttgggaaagaacttgga

SEQ ID NO: 10 - DNA, 18 bp, Artificial Sequence
  YFP - R primer sequence
ccgtggttggcttggtct

SEQ ID NO: 11 - DNA, 15 bp, Artificial Sequence
  YFP - FAM probe sequence
cactccccactgcct

SEQ ID NO: 12 - DNA, 24 bp, Artificial Sequence
  GMS116 - F primer sequence
gtaatatgggctcagaggaatggt

SEQ ID NO: 13 - DNA, 23 bp, Artificial Sequence
  GMS116 - R primer sequence
atggagaagaacattggaattgc

SEQ ID NO: 14 - DNA, 23 bp, Artificial Sequence
  GMS116 - FAM probe sequence
ccatggcccggtaccatctggtc